SEQUENCE LISTING (8 sequences)

SEQ ID NO: 1 - protein, 18 aa, Unknown
  Description of Unknown Conserved prion disease peptide
HTVTTTTKGENFTETDIK

SEQ ID NO: 2 - protein, 26 aa, Unknown
  Description of Unknown Conserved prion disease peptide
HGGGGWGQGGTHGQWNKPSKPKTNMK

SEQ ID NO: 3 - protein, 15 aa, Unknown
  Description of Unknown Conserved prion disease peptide
HSQWNKPSKPKTNMK

SEQ ID NO: 4 - protein, 11 aa, Unknown
  Description of Unknown Conserved prion disease peptide
KPSKPKTNMKH

SEQ ID NO: 5 - protein, 10 aa, Unknown
  Description of Unknown Conserved prion disease peptide
KQHTVTTTTK

SEQ ID NO: 6 - protein, 8 aa, Unknown
  Description of Unknown Conserved prion disease peptide
KPKTNMKH

SEQ ID NO: 7 - protein, 14 aa, Unknown
  Description of Unknown Conserved prion disease peptide
HFFFAKLNCRLYRK

SEQ ID NO: 8 - protein, 14 aa, Unknown
  Description of Unknown Conserved prion disease peptide
KFDTISEKTSDQIH

(19) United States
(12) Patent Application Publication
(10) Pub. No.: US 2015/0147346 A1
(43) Pub. Date: May 28, 2015

(54) REPLIKIN SEQUENCES AND THEIR ANTIBODIES FOR DIAGNOSTICS, THERAPEUTICS, AND VACCINES AGAINST PRION AND NEURODEGENERATIVE DISORDERS INCLUDING ALZHEIMER'S DISEASE

(71) Applicants: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(72) Inventors: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(21) Appl. No.: 14/398,376

(22) PCT Filed: May 1, 2013
(86) PCT No.: PCT/US13/39111

§ 371 Date: Oct. 31, 2014

Related U.S. Application Data
(60) Provisional application No. 61/687,818, filed on May 2, 2012.

(51) Int. Cl. C07K 14/47; C07K 7/06; C07K 7/08

(52) U.S. Cl. CPC C07K 14/47; C07K 7/08; C07K 7/06; A61K 39/00
USPC 424/185.1; 530/324; 530/326; 530/327; 530/328

(57) ABSTRACT

The present invention provides methods of diagnosing, treating, and preventing prion and neurodegenerative disorders including vaccines against prion diseases and neurodegenerative disorders.

This application claims benefit of U.S. Provisional Appln. Ser. No. 61/687,818, filed May 2, 2012.

This application incorporates by reference in their entireties, the following applications: U.S. Provisional Appln. Ser. No. 61/779,324, filed Mar. 13, 2013, U.S. application Ser. No. 13/791,609, filed Mar. 8, 2013, PCT/US2013/030013, filed Mar. 8, 2013, U.S. Provisional Appln. Ser. No. 61/765,106, filed Feb. 15, 2013, U.S. Provisional Appln. Ser. No. 61/724,538, filed Nov. 9, 2012, U.S. application Ser. No. 13/553,137, filed Jul. 19, 2012, PCT/US2012/047451, filed Jul. 19, 2012, U.S. Provisional Appln. Ser. No. 61/609,074, filed Mar. 9, 2012, U.S. application Ser. No. 12/581,112, filed Oct. 16, 2009, U.S. Provisional Appln. Ser. No. 61/246,006, filed Sep. 25, 2009, U.S. application Ser. No. 12/538,027, filed Aug. 7, 2009, U.S. Provisional Appln. Ser. No. 61/185,160, filed Jun. 8, 2009, U.S. Provisional Appln. Ser. No. 61/179,686, filed May 19, 2009, U.S. Provisional Appln. Ser. No. 61/172,115, filed Apr. 23, 2009, U.S. application Ser. No. 12/429,044, filed Apr. 23, 2009, PCT/US09/41565, filed Apr. 23, 2009, and U.S. Provisional Appln. Ser. No. 61/143,618, filed Jan. 9, 2009, U.S. Provisional Appln. Ser. No. 61/087,354, filed Aug. 8, 2008, U.S. Provisional Appln. Ser. No. 61/054,010, filed May 16, 2008, U.S. application Ser. No. 12/108,458, filed Apr. 23, 2008, PCT/US2008/61336, filed Apr. 23, 2008, U.S. application Ser. No. 12/010,027, filed Jan. 18, 2008, U.S. Provisional Appln. Ser. No. 60/991,676, filed Nov. 30, 2007, U.S. application Ser. No. 11/923,559, filed Oct. 24, 2007, now U.S. Pat. No. 8,050,871, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, U.S. Provisional Appln. Ser. No. 60/880,966, filed Jan. 18, 2007, U.S. Provisional Appln. Ser. No. 60/853,744, filed Oct. 24, 2006, U.S. application Ser. No. 11/355,120, filed Feb. 16, 2006, now U.S. Pat. No. 7,894,999, U.S. application Ser. No. 11/116,203, filed Apr. 28, 2005, now U.S. Pat. No. 7,774,144, U.S. application Ser. No. 10/860,050, filed Jun. 4, 2004, now U.S. Pat. No. 7,442,761, U.S. application Ser. No. 10/189,437, filed Jul. 8, 2002, now U.S. Pat. No. 7,452,963, U.S. application Ser. No. 10/105,232, filed Mar. 26, 2002, now U.S. Pat. No. 7,189,800, U.S. application Ser. No. 09/984,057, filed Oct. 26, 2001, now U.S. Pat. No. 7,420,028, and U.S. application Ser. No. 09/984,056, filed Oct. 26, 2001, now U.S. Pat. No. 7,176,275.

### SEQUENCE LISTING

The instant application contains a Sequence Listing, which has been submitted in ASCII format via EFS-Web and is hereby incorporated by reference in its entirety. Said ASCII copy, created on May 1, 2013, is named 13795-48376_SL.txt and is 2,509 bytes in size.

### FIELD OF THE INVENTION

The present invention relates to diagnostics, therapeutics, and vaccines against prion and other neurodegenerative disorders.

### BACKGROUND OF THE INVENTION

Prion diseases and neurodegenerative disorders include, for example, Creutzfeldt-Jakob Disease, Variant Creutzfeldt-Jakob Disease, Gerstmann-Straussler-Scheinker Syndrome, fatal familial insomnia, Kuru, bovine spongiform encephalopathy, chronic wasting disease, scrapie, transmissible mink encephalopathy, feline spongiform encephalopathy, ungulate spongiform encephalopathy, Alzheimer's disease, Parkinson's disease, Huntington's disease, and other neurodegenerative and prion diseases.

Prion diseases generally are known as transmissible spongiform encephalopathies (TSEs). They are presently known to include a family of rare progressive neurodegenerative disorders that affect both humans and animals. Generally, these diseases are distinguished by long incubation periods, characteristic spongiform changes associated with neuronal loss, and a failure to induce inflammatory responses. Currently understood prion diseases in humans include, for example, Creutzfeldt-Jakob Disease (CJD), Variant Creutzfeldt-Jakob Disease (vCJD), Gerstmann-Straussler-Scheinker Syndrome, Fatal Familial Insomnia, and Kuru. Currently understood prion diseases in animals include, for example, bovine spongiform encephalopathy (BSE), chronic wasting disease (CWD), scrapie, transmissible mink encephalopathy, feline spongiform encephalopathy, ungulate and spongiform encephalopathy.

Neurodegenerative diseases include, for example, Alzheimer's disease, Parkinson's disease, and Huntington's disease. Neurodegeneration is generally considered to result from progressive loss of structure or function of neurons, including death of neurons. Researchers generally currently believe similarities among neurodegenerative diseases relate to degeneration at the sub-cellular level, including atypical protein assemblies and induced cell death.

Serine protease inhibitors, known as serpins, are understood to contribute to Alzheimer's disease. Nielsen et al. measured plasma and CSF levels of serpins in patients with dementia and found levels of CSF neuroserpin higher in sufferers of Alzheimer's disease as compared to controls. Neilsen et al., “Plasma and CSF serpins in Alzheimer disease and dementia with Lewy bodies,” Neurology 2007 Oct. 16; 69(16):1569-79). They found higher levels of serpins (along with alpha(1)-antichymotrypsin) facilitated diagnostic classification in Alzheimer's disease. Fabbro et al., found neuroserpin inhibits tissue plasminogen activator activity in the brains of sufferers from Alzheimer's disease. Fabbro et al., “Plasminogen activator activity is inhibited while neuroserpin is up-regulated in the Alzheimer disease brain,” J Neurochem. 2009 April; 109(2):303-15. They considered that increased levels of neuroserpin inhibits tissue plasminogen activator activity, which leads to reduced plasmin and may be responsible for reduced clearance of amyloid-beta from the brain. Amyloid-beta plaques are a pathological hallmark of Alzheimer's disease. Belorgey et al. further found pH-dependent stability of neuroserpin to be mediated by histidines. Belorgey et al., “pH-dependent stability of neuroserpin is mediated by histidines 119 and 138; implications for the control of beta-sheet A and polymerization,” Protein Sci. 2010 February; 19(2):220-8. Researchers have until now unsuccessfully attempted to determine what is responsible for the upregulation of neuroserpin in Alzheimer's disease.

There are currently no therapies to cure or stop the progression of prion diseases or other neurodegenerative diseases. Additionally, diagnostic methods for determining the presence and progression of neurodegenerative diseases remain limited in accuracy and precision. Further, the cause of many prion diseases and other degenerative diseases remain unknown.

Researchers have until now not known how to design therapies against prion diseases. One impediment to the design of therapies has been the absence of knowledge of antigens present in prion diseases. Further, no real structure for targeting prions has until now been identified. As a result, no vaccines (or other therapies) could be developed against these diseases or against the mechanisms of these diseases since no structural targets had been identified for controlling infectivity and replication in these diseases.

The art, therefore, is in real need of diagnostic, therapeutic, and preventive methods and compounds to antagonize or halt progression or initiation of these diseases by targeting structures in prion diseases related to infectivity and replication.

How prions might be infective has continued to be a matter of great controversy in the art. For example, researchers have asked how a prion can be infective without direct involvement of nucleic acid replication. In direct response to this lack of knowledge in the art, the applicants specifically set out to discover a marker of infectivity in amino acid sequences involved in prion diseases.

Replikin peptides are a family of small peptides that have been correlated with the phenomenon of rapid replication in influenza, malaria, West Nile virus, foot and mouth disease, and many other pathogens. Replikin peptides have likewise been generally correlated with the phenomenon of rapid replication in viruses, organisms, and malignancies.

Identification of Replikin peptides has provided targets for detection and treatment of pathogens and cancers, including vaccine development against virulent pathogens such as influenza virus, malaria, West Nile virus, and foot and mouth disease virus. In general, knowledge of and identification of this family of peptides enables development of effective therapies and vaccines for pathogens that harbor Replikins. The phenomenon of the association of Replikins with rapid replication and virulence has been fully described in, for example, U.S. Pat. No. 7,189,800; U.S. Pat. No. 7,176,275; U.S. Pat. No. 7,442,761; U.S. Pat. No. 7,894,999, U.S. Pat. No. 8,050,871, and U.S. application Ser. No. 12/108,458. Both Replikin concentration (number of Replikin sequences per 100 amino acids) and Replikin composition have been correlated with the functional phenomenon of rapid replication.

Research by the applicants over the last two decades has revealed amino acid sequences associated with rapid replication in a biology-wide array of organisms including pathogenic organisms such as viruses and non-pathogenic organisms such as plants grown for food and various species of algae. These sequences have been called Replikin sequences.

Research has revealed Replikin sequences in pathogens related to rapid replication, infectivity, and lethality. See, e.g., WO 2008/143717. Examples of such pathogens include influenza virus, malaria, porcine respiratory and reproductive syndrome virus, e. coli, West Nile virus, foot and mouth disease virus, anthrax, small pox virus, coronaviruses (including SARS virus), porcine circovirus, taura syndrome virus in shrimp, white spot syndrome virus in shrimp, as well as other viruses and non-virus pathogens. Replikin sequences have been shown to be conserved in these organisms and to relate to the organisms' survival. Research has further revealed that the presence of Replikin sequences correlates with outbreaks of viruses and other pathogens, including increases in lethality and rate of replication. See, e.g., WO 2010/132209. In September of 2011, the United Nations Food and Agricultural Organization (FAO) warned the health community that concentrations of Replikin sequences (REPLIKIN COUNTS) at their highest levels in fifty years in the H1N1 and H5N1 strains of influenza A virus are a danger sign. See, e.g., VeterinaryNews.DVM360.com (Sep. 12, 2011). Outbreaks of H1N1 and H5N1 strains of influenza A virus continue to occur.

Identification of Replikin peptide sequences in prion and neurodegenerative diseases as provided in an aspect of the present invention responds to a long felt need in the art for diagnostic, therapeutic, and preventive methods and compounds to antagonize or halt progression or initiation of prion and other neurodegenerative diseases.

### SUMMARY OF THE INVENTION

The present invention provides methods and substances for diagnosing, preventing, and treating prion and neurodegenerative disorders including immunogenic compositions and vaccines against prion diseases and neurodegenerative disorders.

A first non-limiting aspect of the disease provides an immunogenic composition comprising a Replikin peptide sequence identified in a prion disease or a neurodegenerative disease or a homologue of a Replikin peptide sequence identified in a prion disease or neurodegenerative disease or an antigenic fragment of a Replikin peptide sequence or homologue of a Replikin peptide sequence identified in a prion disease or neurodegenerative disease. In a non-limiting embodiment of the first aspect of the invention, the Replikin peptide sequence identified in the prion disease or the neurodegenerative disease may be part of a body affected or changed by the disease. In another non-limiting embodiment, the part of a body affected or changed by a prion disease or a neurodegenerative disease is a protein, protein fragment, polypeptide, or peptide. The part of a body affected or changed by a prion disease or a neurodegenerative disease can include, for example, a protein, protein fragment, polypeptide, peptide, or any tissue affected or changed by the disease, including a change in the three-dimensional structure of the protein, protein fragment, polypeptide, or peptide, such as a change in folding.

In a non-limiting embodiment, a Replikin peptide sequence may be conserved. In a non-limiting embodiment, a Replikin peptide sequence may be conserved in various prion diseases and/or neurodegenerative diseases, may be conserved across time in one or more prion and/or neurodegenerative diseases, or may be conserved across time and across prion diseases and neurodegenerative diseases.

In a non-limiting embodiment, a prion disease or a neurodegenerative disease may be any prion disease or neurodegenerative disease. In a non-limiting embodiment, the prion disease or neurodegenerative disease may be Creutzfeldt-Jakob Disease, Variant Creutzfeldt-Jakob Disease, Gerstmann-Straussler-Scheinker Syndrome, Fatal Familial Insomnia, Kuru, bovine spongiform encephalopathy, chronic wasting disease, scrapie, transmissible mink encephalopathy, feline spongiform encephalopathy, ungulate spongiform encephalopathy, Alzheimer's disease, Parkinson's disease, or Huntington's disease.

In a non-limiting embodiment of the first aspect of the invention, a homologue of a Replikin peptide sequence may be comprised in a protein, protein fragment, polypeptide, or peptide and the homologue may be at least 30%, 40%, 50%, 60%, 70%, 80%, 90%, 95%, 97%, 98%, or 99% or more homologous with a Replikin peptide sequence identified in a prion disease or a neurodegenerative disease. In another non-limiting embodiment, the homologue may share the same lysine residues and histidine residue present in the Replikin peptide sequence identified in a prion disease or neurodegenerative disorder.

In another non-limiting embodiment, an immunogenic composition may comprise a peptide consisting essentially of a Replikin peptide sequence identified in a prion disease or a neurodegenerative disease or a peptide that consists of a Replikin peptide sequence identified in a prion disease or a neurodegenerative disease.

In another non-limiting embodiment, the immunogenic composition may comprise a protein, protein fragment, polypeptide, or peptide comprising at least one peptide sequence of SEQ ID NO(s): 1-8, at least one homologue of SEQ ID NO(s): 1-8 that may be at least 30%, 40%, 50%, 60%, 70%, 80%, 90%, 95%, 97%, 98%, or 99% or more homologous any one of SEQ ID NO(s): 1-8, at least one peptide sharing the same lysine residues and histidine residue creating the Replikin structure of SEQ ID NO(s): 1-8, at least one peptide consisting essentially of SEQ ID NO(s): 1-8, at least one peptide consisting of any one of SEQ ID NO(s): 1-8, or at least one antigenic fragment of at least one of SEQ ID NO(s): 1-8. In a non-limiting embodiment, the immunogenic composition may comprise a peptide sequence consisting essentially of 7 to 50 amino acid residues and comprising at least one of SEQ ID NO(s): 1-8 or a homologue of any one of SEQ ID NO(s): 1-8.

A second non-limiting aspect of the present invention provides, a vaccine comprising at least one of the immunogenic compositions listed above and/or disclosed herein. In a non-limiting embodiment of the second aspect of the invention, a vaccine may comprise a Replikin peptide sequence identified in a prion disease or a neurodegenerative disease or a homologue of a Replikin peptide sequence identified in a prion disease or neurodegenerative disease or an antigenic fragment of a Replikin peptide sequence or homologue of a Replikin peptide sequence. In another non-limiting embodiment, the Replikin peptide sequence identified in the prion disease or the neurodegenerative disease may be part of a body affected or changed by the disease. In another non-limiting embodiment, the part of a body affected or changed by a prion disease or a neurodegenerative disease is a protein, protein fragment, polypeptide, or peptide. The prion disease or neurodegenerative disease may be any prion disease or neurodegenerative disease. In a non-limiting embodiment, the prion disease or neurodegenerative disease may be Creutzfeldt-Jakob Disease, Variant Creutzfeldt-Jakob Disease, Gerstmann-Straussler-Scheinker Syndrome, Fatal Familial Insomnia, Kuru, bovine spongiform encephalopathy, chronic wasting disease, scrapie, transmissible mink encephalopathy, feline spongiform encephalopathy, ungulate spongiform encephalopathy, Alzheimer's disease, Parkinson's disease, or Huntington's disease.

In a non-limiting embodiment of the second aspect of the invention, a homologue of a Replikin peptide sequence may be comprised in a protein, protein fragment, polypeptide, or peptide and the homologue may be at least 30%, 40%, 50%, 60%, 70%, 80%, 90%, 95%, 97%, 98%, or 99% or more homologous with a Replikin peptide sequence identified in a prion disease or a neurodegenerative disease. In another non-limiting embodiment, the homologue may share the same lysine residues and histidine residue present in the Replikin peptide sequence identified in a prion disease or neurodegenerative disease.

In another non-limiting embodiment of the second aspect of the invention, a vaccine may comprise a peptide consisting essentially of a Replikin peptide sequence identified in a prion disease or a neurodegenerative disease or a peptide that consists of a Replikin peptide sequence identified in a prion disease or a neurodegenerative disease.

In a non-limiting embodiment, a vaccine may comprise more than one Replikin peptide sequence, homologue of a Replikin peptide sequence, peptide sequence sharing the structure of lysine residues and histidine residue that define a Replikin sequence, antigenic fragment of a Replikin peptide sequence, or antigenic fragment of a homologue of a Replikin peptide sequence.

In a non-limiting embodiment, a vaccine may comprise a protein, protein fragment, polypeptide, or peptide comprising at least one peptide sequence of SEQ ID NO(s): 1-8, at least one homologue of SEQ ID NO(s): 1-8 that is at least 30%, 40%, 50%, 60%, 70%, 80%, 90%, 95%, 97%, 98%, or 99% or more homologous with at least one of SEQ ID NO(s): 1-8, at least one peptide sharing the same lysine residues and histidine residue creating the Replikin structure of SEQ ID NO(s): 1-8, at least one peptide consisting essentially of SEQ ID NO(s): 1-8, at least one peptide consisting of any one of SEQ ID NO(s): 1-8, or at least one antigenic fragment of SEQ ID NO(s): 1-8.

A non-limiting vaccine may comprise at least one non-limiting pharmaceutically-acceptable carrier, excipient, adjuvant, other additional component, or combination thereof.

A non-limiting embodiment provides an equal-parts-by-weight mixture of each of SEQ ID NO(s): 1-8. The mixture may be comprised in sterile water.

A third non-limiting aspect of the present invention provides a method of making a vaccine against a prion disease or other neurodegenerative disease comprising identifying at least one Replikin peptide sequence or fragment of a Replikin peptide sequence in a protein, protein fragment, polypeptide, or peptide expressed in a part of a body affected or changed by the disease and making a vaccine comprising said protein, protein fragment, polypeptide, or peptide comprising said Replikin peptide sequence, a homologue of said Replikin peptide sequence, or a fragment of said Replikin peptide sequence. In a non-limiting embodiment, the vaccine may comprise a plurality of Replikin peptide sequences, fragments of Replikin peptide sequences, or homologues of Replikin peptide sequences. In a non-limiting embodiment, the vaccine may comprise at least one peptide sequence of SEQ ID NO(s): 1-8, at least one fragment of SEQ ID NO(s): 1-8, or at least one homologue of SEQ ID NO(s): 1-8 that is at least 30%, 40%, 50%, 60%, 70%, 80%, 90%, 95%, 97%, 98%, or 99% or more homologous with at least one of SEQ ID NO(s): 1-8. In a further non-limiting embodiment, a vaccine may comprise a mixture of peptides of each of SEQ ID NO(s): 1-8.

A fourth non-limiting aspect of the invention provides an isolated or synthesized protein, protein fragment, polypeptide, or peptide comprising a Replikin peptide sequence identified in a part of a body affected or changed by a prion disease or neurodegenerative disease, a homologue of a Replikin peptide sequence identified in a part of a body a body affected or changed by a prion disease or neurodegenerative disease, wherein said homologue is at least 30%, 40%, 50%, 60%, 70%, 80%, 90%, 95%, 97%, 98%, or 99% or more homologous with a Replikin peptide sequence identified in a prion disease or a neurodegenerative disease, a peptide sharing the same lysine residues or histidine residue that define a Replikin peptide sequence identified in a part of a body a body affected or changed by a prion disease or neurodegenerative disease, or an antigenic fragment of a Replikin peptide sequence identified in a part of a body a body affected or changed by a prion disease or neurodegenerative disease. Another non-limiting embodiment provides an isolated or synthesized peptide that is an antigenic fragment of a Replikin peptide sequence identified in a part of a body a body affected or changed by a prion disease or neurodegenerative disease.

In a non-limiting embodiment, the isolated or synthesized protein, protein fragment, polypeptide, or peptide may consist essentially of a Replikin peptide sequence or homologue of a Replikin peptide sequence. Another non-limiting embodiment provides an isolated or synthesized peptide consisting of a Replikin peptide sequence or homologue of a Replikin peptide sequence. In a non-limiting embodiment, a Replikin sequence may be at least one of SEQ ID NO(s): 1-8. In a non-limiting embodiment, a peptide may consist essentially of at least one of SEQ ID NO(s): 1-8 or may consist of at least one of SEQ ID NO(s): 1-8.

In a non-limiting embodiment, an isolated or synthesized peptide sequence may consist essentially of 7 to 50 amino acid residues and comprise at least one of SEQ ID NO(s): 1-8 or a homologue of any one of SEQ ID NO(s): 1-8.

A fifth non-limiting aspect of the present invention provides a binding molecule that preferentially binds at least one Replikin peptide sequence identified in a prion disease or a neurodegenerative disease, at least one homologue of Replikin peptide sequence identified in a prion disease or a neurodegenerative disease, or at least one antigenic fragment of a Replikin peptide sequence identified in a prion disease or a neurodegenerative disease. In a non-limiting embodiment, the Replikin peptide sequence identified in a prion disease or a neurodegenerative disease may be part of a body affected or changed by the disease.

In a non-limiting embodiment, a binding molecule may be any immunogenic binding molecule or any molecule capable of preferentially binding a Replikin peptide sequence or a fragment of a Replikin peptide sequence. In a non-limiting embodiment, a Replikin peptide sequence or a fragment of the Replikin peptide sequence may be individually isolated or may be present in a larger molecule. In a non-limiting embodiment, a binding molecule may be an antibody, an antibody fragment, an Fab fragment, an Fc fragment, or any binding portion of an antibody.

In a non-limiting embodiment, a binding molecule may preferentially bind at least one of SEQ ID NO(s): 1-8, at least one homologue of SEQ ID NO(s): 1-8 that is at least 30%, 40%, 50%, 60%, 70%, 80%, 90%, 95%, 97%, 98%, or 99% or more homologous with at least one of SEQ ID NO(s): 1-8, at least one sequence that shares the lysine residues and histidine residue that define the Replikin peptide sequence of SEQ ID NO(s): 1-8, or at least one fragment of any one of SEQ ID NO(s): 1-8.

A sixth non-limiting aspect of the present invention provides a method of diagnosing a prion disease or a neurodegenerative disease comprising identifying a Replikin peptide sequence or homologue of a Replikin peptide sequence identified in a prion disease or a neurodegenerative disease. A non-limiting embodiment of the sixth aspect of the present invention provides a method of diagnosing a prion or other neurodegenerative disease comprising binding a binding molecule to a Replikin peptide or homologue or fragment of a Replikin peptide in a sample. In a further non-limiting embodiment, a binding molecule binds the peptide or fragment in a tissue specimen of a subject, including, for example, in the blood of a subject.

A seventh non-limiting aspect of the present invention provides an isolated or synthesized nucleic acid sequence that encodes a protein, protein fragment, polypeptide, or peptide comprising at least one Replikin peptide sequence (or at least one homologue of at least one Replikin peptide sequence) identified in a prion disease or a neurodegenerative disease. In a non-limiting embodiment, the nucleic acid sequence encodes a protein fragment, polypeptide, or peptide comprising at least one peptide sequence that is at least 30%, 40%, 50%, 60%, 70%, 80%, 90%, 95%, 97%, 98%, or 99% or more homologous with at least one of SEQ ID NO(s): 1-8. In a non-limiting embodiment, the isolated or synthesized nucleic acid sequence encodes for a peptide consisting essentially of 7 to 50 amino acid residues and comprising any one or more of the peptide sequences of SEQ ID NO(s): 1-8. In another non-limiting embodiment of the seventh aspect of the present invention, the isolated or synthesized nucleic acid sequence is comprised in an immunogenic compound. In a non-limiting embodiment, the immunogenic composition is a therapeutic compound. In another non-limiting embodiment, the isolated or synthesized nucleic acid sequence is comprised in a vaccine.

Another non-limiting embodiment of the seventh aspect of the present invention provides an isolated or synthesized nucleic acid sequence that is antisense to a nucleic acid sequence that encodes for a peptide that is at least 30%, 40%, 50%, 60%, 70%, 80%, 90%, 95%, 97%, 98%, or 99% or more homologous with at least one of the Replikin peptide sequence identified in a prion disease or neurodegenerative disease or at least one functional fragment of at least one such Replikin peptide sequence. In a non-limiting embodiment, the Replikin peptide sequence is at least one of SEQ ID NO(s): 1-8. Another non-limiting embodiment provides a small interfering nucleic acid sequence that is about 10 to about 50 nucleic acids in length and is 30%, 40%, 50%, 60%, 70%, 80%, 90%, 95%, 97%, 98%, or 99% or more homologous with a nucleic acid sequence that encodes for any portion of a Replikin peptide sequence identified in a prion disease or a neurodegenerative disease including, for example, SEQ ID NO(s): 1-8. In another non-limiting embodiment the small interfering nucleic acid sequences is about 15 to about 45, about 20 to about 30, or about 21, 22, 23, 24, 25, 26, 27, 28, or 29 nucleic acids in length.

### DETAILED DESCRIPTION OF THE INVENTION

### Definitions

A “protein fragment” as used in this specification is any portion of an expressed whole protein. A protein fragment may reflect an expressed whole protein with one or more amino acids removed from the amino acid sequence of the expressed whole protein. A protein fragment may also reflect an amino acid sequence that is at least 30%, 40%, 50%, 60%, 70%, 80%, 90%, 95%, 95%, 97%, 98%, or 99% or more homologous with any portion of an expressed whole protein. A “polypeptide,” as used in this specification, is any portion of a protein fragment and is less than an expressed whole protein. A portion of a whole protein is less than the whole protein and a portion of a protein fragment is less than the protein fragment. A portion may comprise at least one amino acid residue less than a whole.

“Homologous” or “homology” as used in this specification indicate that an amino acid sequence or nucleic acid sequence exhibits substantial structural equivalence with another sequence, namely, any Replikin peptide sequence (including, but not limited to, SEQ ID NO(s): 1-8) identified in a prion or other neurodegenerative disease or any nucleotide sequence encoding a Replikin peptide sequence in a prion or other neurodegenerative disease (a redundancy in a coding sequence may be considered identical to a sequence encoding the same amino acid residue).

To determine the percent identity or percent homology of an identified sequence, the sequence is aligned for optimal comparison purposes with any one of identified basis sequences. Where gaps are necessary to provide optimal alignment, gaps may be introduced in the identified sequence or in the basis sequence. Gaps may be within the sequence and/or on the end or ends of the sequence. When a position in the identified sequence is occupied by the same amino acid residue or same nucleotide as the corresponding position in the basis sequence, the molecules are considered identical at that position (as used herein amino acid or nucleic acid “identity” is equivalent to amino acid or nucleic acid “homology”). To determine percent homology, the amino acid residues or nucleotides at corresponding amino acid positions or nucleotide positions are compared between the identified sequence and the basis sequence. The total number of amino acid residues or nucleotides in the identified sequence that are identical with amino acid residues or nucleotides in the basis sequence is divided by the total number of residues or nucleotides in the basis sequence with the addition of gaps (if the number of residues or nucleotides in the basis sequence is greater than the total number of residues or nucleotides in the identified sequence) or by the total number of amino acid residues or nucleotides in the identified sequence with the addition of gaps (if the number of residues or nucleotides in the identified sequence is greater than the total number of residues or nucleotides in the basis sequence). The final number is determined as a percentage. As such, the percent identity between the two sequences is a function of the number of identical positions shared by the sequences, taking into account the number of gaps (where a gap must be introduced for optimal alignment of the two sequences) and the length of each gap. Any structural or functional differences between sequences having sequence identity or homology will not affect the ability of the sequence to function as indicated in the desired application.

For example, SEQ ID NO: 1 (HTVTTTTKGENFTETDIK) is considered 40% homologous with SEQ ID NO: 5 (KQHTVTTTTK). The 40% homology is determined as follows: SEQ ID NO: 5 is the identified sequence. SEQ ID NO: 1 is the basis sequence. Upon alignment, SEQ ID NO: 5 is identical to SEQ ID NO: 1 at positions 1-8 of SEQ ID NO: 1. Two additional gap positions are included in SEQ ID NO: 1 on the C-terminus since K and Q at the C-terminus of SEQ ID NO: 5 do not share identity with the residues at the C-terminus of SEQ ID NO: 1. To determine percent homology, then, the 8 aligned identical residues are divided by the total number of residues in SEQ ID NO: 1 (18 residues) with the addition of two residues for the gap added at the C-terminus of SEQ ID NO: 1. The total number of residues is considered to be 20. Dividing 8 by 20 provides 0.40 or 40% homology between SEQ ID NO: 1 and SEQ ID NO: 5. Likewise, SEQ ID NO: 4 is considered more than 72% homologous with SEQ ID NO: 6 where SEQ ID NO: 4 is the basis sequence and SEQ ID NO: 6 is the identified sequence.

As used herein an “immunogenic fragment,” “antigenic fragment,” or “functional fragment” of a Replikin peptide sequence or a homologue of a Replikin peptide sequence is a fragment that provides at least a portion of cross-reactivity with an antibody or antibody fragment against the Replikin sequence.

As used herein a “vaccine” is any substance, compound, composition, mixture, or other therapeutic substance that, when administered to a human or animal via any method of administration known to the skilled artisan now or hereafter, produces an immune response, an antibody response, a blocking response, or a protective effect in the human or animal.

As used herein, a “Replikin sequence” is an amino acid sequence of 7 to about 50 amino acid residues comprising (1) a first lysine residue located six to ten residues from a second lysine residue, (2) at least one histidine residue; and (3) at least 6% lysine residues. A Replikin sequence may have a lysine residue on one end of the sequence and a lysine residue or histidine residue on the other end of the sequence. For the purpose of determining Replikin concentration, a Replikin sequence is the shortest amino acid sequence of 7 to 50 amino acid residues comprising (1) a first lysine residue located six to ten residues from a second lysine residue, (2) at least one histidine residue; and (3) at least 6% lysine residues. A Replikin sequence may comprise any number of lysine residues and any number of histidine residues so long as any two lysine residues and any one histidine residue reflect the requirements of the Replikin sequence. As a result, a Replikin sequence counted as part of the Replikin concentration of a sequence of amino acid residues may comprise overlapping Replikin sequences.

The term “Replikin sequence” may also refer to a nucleic acid sequence encoding an amino acid sequence having 7 to about 50 amino acids comprising:
(1) at least one lysine residue located six to ten amino acid residues from a second lysine residue;
(2) at least one histidine residue; and
(3) at least 6% lysine residues,
   wherein the amino acid sequence may comprise a terminal lysine and may further comprise a terminal lysine or a terminal histidine or may be the shortest amino acid sequence of 7 to 50 amino acid residues comprising (1) a first lysine residue located six to ten residues from a second lysine residue, (2) at least one histidine residue; and (3) at least 6% lysine residues.

As used herein, the term “peptide” or “protein” refers to a compound of two or more amino acids in which the carboxyl group of one amino acid is attached to an amino group of another amino acid via a peptide bond.

As used herein, an “isolated” peptide may be synthesized by organic chemical methods. An isolated peptide may also be synthesized by biosynthetic methods. An isolated peptide also may refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silico from nucleic acid or amino acid sequences that are available through public or private databases or sequence collections. An isolated peptide may be synthesized by biosynthetic or organic chemical methods.

Proteins, protein fragments, polypeptides, or peptides in this specification may be chemically synthesized by any method known to one of skill in the art now and hereafter. For example, isolated proteins, protein fragments, polypeptides, or peptides may be synthesized by solid phase synthesis. The production of these materials by chemical synthesis avoids the inclusion of (or the need to remove by purification) materials that are byproducts of other production methods such as recombinant expression or isolation from biological material. Such byproducts may include, for example, avian proteins associated with vaccines produced using birds' eggs or bacterial proteins associated with recombinant production in bacteria.

An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well-known in the art that, due to redundancy in the genetic code, individual nucleotides can be readily exchanged in a codon and still result in an identical amino acid sequence. As will be understood by one of ordinary skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

As used herein, “Replikin Count” or “Replikin concentration” refers to the number of Replikin sequences per 100 amino acid residues in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence, wherein a Replikin sequence is a peptide of 7 to 50 amino acid residues comprising (1) a first lysine residue six to ten residues from a second lysine residue, (2) at least one histidine residue, (3) and 6% or more lysine residues where the Replikin sequence is the shortest sequence comprising the first and second lysine residues of element (1) and the at least one histidine residue of element (2). A Replikin sequence for the purpose of determining Replikin concentration as described in this paragraph may also be a nucleic acid that encodes a Replikin peptide sequence defined according to this paragraph.

### Immunogenic Compositions and Vaccines Against Prion Diseases

One non-limiting aspect of the present invention provides immunogenic compositions and vaccines against a prion or other neurodegenerative disease. An immunogenic composition and/or vaccine may comprise any Replikin peptide sequence or homologue identified in a prion disease. Such prion diseases may include, and are not limited to, for example, Creutzfeldt-Jakob Disease, Variant Creutzfeldt-Jakob Disease, Gerstmann-Straussler-Scheinker Syndrome, Fatal Familial Insomnia, Kuru, bovine spongiform encephalopathy, chronic wasting disease, scrapie, transmissible mink encephalopathy, feline spongiform encephalopathy, ungulate spongiform encephalopathy, Alzheimer's disease, Parkinson's disease, Huntington's disease, or any other prion or neurodegenerative disease known to one of skill in the art now or hereafter.

Until now, no vaccines against prion diseases or other neurodegenerative diseases were known in the art. Researchers did not know how to design therapies against prion diseases significantly in part because researchers were not aware of targets within prion diseases that were antigenic and related to replication and infectivity. No real structure for controlling prion replication and infectivity was available.

The applicants intensively studied prion diseases and searched for Replikin sequences. Beginning with the discovered Replikin sequence antigens, the applicants discovered that the antigens provide a target for controlling replication and infectivity in prion diseases.

Increasing concentrations of Replikin sequences have been shown by the applicants to provide warnings of outbreaks of pathogens. See, e.g., US 2009/0017052. These Replikin Count warnings have been commended as accurate and useful by the United Nations Food and Agriculture Organization (FAO) in alerting the public to outbreaks. See, e.g., VeterinaryNews.DVM360.com (Sep. 12, 2011). The applicants have also previously demonstrated that Replikin sequences are conserved, antigenic, related to replication and lethality, and good targets for vaccines. See, id. Replikin sequences are associated with rapid replication in a biology-wide array of organisms including pathogenic organisms such as viruses and non-pathogenic organisms such as plants grown for food and various species of algae. Examples of pathogens in which Replikin sequences provide warnings of outbreaks include influenza virus, malaria, porcine respiratory and reproductive syndrome virus, e. coli, West Nile virus, foot and mouth disease virus, anthrax, small pox virus, coronaviruses (including SARS virus), porcine circovirus, taura syndrome virus in shrimp, white spot syndrome virus in shrimp, as well as other virus and non-virus pathogens.

One of skill in the art would not have known or expected that Replikin sequences would provide targets for controlling replication and lethality in prion diseases and would not have been motivated to research Replikin sequences as a possibility for targets for controlling replication and lethality. In fact, until the present discovery, the art continued to experience great controversy over how amino acid sequences in prion diseases could even be infectious.

In contrast to this established understanding in the art, the applicants specifically sought out and discovered targets within prion diseases that were markers of infectivity and lethality. The applicants further researched and discovered these markers were antigenic and provided targets for antibodies capable of controlling replication. These targets were also researched and discovered to be conserved, providing for therapies across prion diseases.

One of ordinary skill in the art would not have expected Replikin sequences to be targets in prion diseases because the machinery of replication in prion diseases continues to be unknown and is particularly and significantly different from all other known pathogens. Further, the absence of nucleic acid translation in the pathogenic process in prion diseases provides a completely different mechanism for replication and infection. As a result, one of ordinary skill would not have predicted that Replikin sequences could serve as targets for vaccines in prion diseases and would not have foreseen a benefit in or been motivated to undertake the extensive further research of the applicants to discover Replikin sequences as targets for vaccines in prion disease.

### Exemplary Immunogenic Composition and Vaccine Against Prion and Other Neurodegenerative Diseases

One non-limiting example of an immunogenic composition and vaccine against prion and other neurodegenerative diseases is an immunogenic composition and vaccine comprising at least one protein, protein fragment, polypeptide, or peptide comprising at least one of

(SEQ ID NO: 1)
HTVTTTTKGENFTETDIK,
(SEQ ID NO: 2)
HGGGGWGQGGTHGQWNKPSKPKTNMK,
(SEQ ID NO: 3)
HSQWNKPSKPKTNMK,
(SEQ ID NO: 4)
KPSKPKTNMKH,
(SEQ ID NO: 5)
KQHTVTTTTK,
(SEQ ID NO: 6)
KPKTNMKH,
(SEQ ID NO: 7)
HFFFAKLNCRLYRK,
and
(SEQ ID NO: 8)
KFDTISEKTSDQIH.

The composition and vaccine may further comprise additional proteins, protein fragments, polypeptides, and/or peptides comprising at least one other peptide of SEQ ID NO(s): 1-8. The composition may further comprise proteins, protein fragments, polypeptides, or peptides comprising at least two peptides of SEQ ID NO(s): 1-8, at least three peptides of SEQ ID NO(s): 1-8, at least four peptides of SEQ ID NO(s): 1-8, or up to at least all eight peptides of SEQ ID NO(s): 1-8.

An immunogenic composition and vaccine may also comprise at least one peptide consisting essentially of at least one peptide of SEQ ID NO(s): 1-8. It may comprise two or more peptides consisting essentially of SEQ ID NO(s): 1-8. It likewise may comprise peptides consisting essentially of three, four, five, six, seven, or all eight peptides of SEQ ID NO(s): 1-8. The immunogenic composition and/or vaccine may likewise comprise at least one of the listed peptides as well as one or more other protein, protein fragment, polypeptide, or peptide comprising any other Replikin peptide identified in a prion disease. It may likewise comprise at least one peptide consisting of at least one sequence of SEQ ID NO(s): 1-8. A non-limiting exemplary vaccine comprises a mixture of each of SEQ ID NO(s): 1-8.

An immunogenic composition may comprise an antigenic fragment of a Replikin peptide sequence identified in a prion disease or neurodegenerative disease and may comprise a mixture of antigenic fragments and/or Replikin peptide sequences. An immunogenic composition may comprise an antigenic fragment of any one or more of SEQ ID NO(s): 1-8.

### Method of Making Vaccine

One aspect of the invention includes methods of making vaccines against prion diseases. One non-limiting method comprises, for example, the steps of (1) identifying a Replikin peptide expressed in a prion or other neurodegenerative disease using any method known to one of skill in the art now or hereafter, (2) making a vaccine comprising the identified Replikin peptide, homologue of the identified Replikin peptide, or fragment of the identified Replikin peptide. The vaccine may comprise, for example, the peptide comprised in a protein in which the Replikin peptide is identified, in a protein fragment, in a larger polypeptide, a peptide consisting essentially of the peptide, a peptide consisting of the identified peptide, any homologue of the peptide, including peptides having the same Replikin structure of the identified peptide, or any fragment of the identified peptide, including an antigenic fragment.

### Polypeptides and Peptides, One Aspect of the Invention

A protein, protein fragment, polypeptide, or peptide of an aspect of the invention comprises any Replikin peptide sequence or homologue thereof identified in a prion or other neurodegenerative disease. A protein, protein fragment, polypeptide, or peptide of an aspect of the invention may comprise, for example, any homologue of any one of SEQ ID NO(s): 1-8. One aspect of the invention is a protein, protein fragment, polypeptide, or peptide comprising the structure of a Replikin peptide. The structure of a Replikin peptide includes a peptide having lysine residues and a histidine residue at about the same residue positions in the peptide as a Replikin peptide identified in a prion disease or other neurodegenerative disease, including a peptide having lysine residues and a histidine residue at about the same residue positions as any one of SEQ ID NO(s): 1-8. In one embodiment, the lysine residues and histidine residue that creates the definition of the Replikin peptide of a prion disease are present in the structure of one of the peptides that are an aspect of the invention.

One aspect of a peptide includes a peptide wherein the Replikin structure is conserved. The structure of a Replikin peptide may be defined, for example, as the lysines and histidine that create the definition of a Replikin peptide. Such peptides, their homologues, and any protein, protein fragment, polypeptide, or peptide comprising said peptide or homologue is useful in an immunogenic composition and/or vaccine that is an aspect of the present invention or useful in diagnosis of a prion or other neurodegenerative disease.

Examples 1-8 below provide examples of the discovery of conserved Replikin peptides useful for diagnosis of prion and other neurodegenerative diseases and as vaccines against prion and other neurodegenerative diseases. Conserved sequences having the antigenic structure of a Replikin peptide provide excellent targets for diagnosing presence of the disease. Further, because Replikin peptides are associated with replication in pathogenesis, targeting such peptides with a vaccine, with passive immunity, with siRNA's or antisense nucleic acids, or with any other attacking methods that target the Replikin peptide or a fragment thereof provides for prevention and control of the disease.

### Antibodies and Antibody Derivatives and Passive Immunity

Another aspect of the invention provides binding agents that bind at least to a functional fragment of a Replikin sequence identified in a prion disease or a neurodegenerative disease. Binding agents are provided including an antibody, antibody fragment, or binding agent that binds to at least a portion of an amino acid sequence of at least one protein, protein fragment, polypeptide, or peptide comprising at least one peptide A, where peptide A is at least 30%, 40%, 50%, 60%, 70%, 80%, 90% or 95%, or 100%, homologous with at least one Replikin peptide identified in a prion disease or a neurodegenerative disease, which may include, for example, at least one Replikin peptide sequence of SEQ ID NO(s): 1-8.

The amino acid sequence of a protein fragment, polypeptide, or peptide of one aspect of the invention may partially match the amino acid sequence of an expressed whole protein where at least one, five, ten, twenty, thirty, forty, fifty, one hundred, two hundred, three hundred, four hundred, five hundred or more amino acid residues of the amino acid sequence of the expressed whole protein are not present in the protein fragment, polypeptide, or peptide. The amino acid sequence of the protein fragment, polypeptide, or peptide may also partially match the amino acid sequence of an expressed whole protein where at least one, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of the amino acid sequence of at least one terminus of the expressed whole protein are not present at least one terminus of said protein fragment, polypeptide, or peptide. Binding agents may bind any of these amino acid sequences.

Binding agents are also provided including an antibody, antibody fragment, or binding agent that binds to at least a portion of an amino acid sequence that is 30%, 40%, 50%, 60%, 70%, 80%, 90%, 95%, 97%, 98%, or 99% or more homologous with at least one Replikin peptide of a prion disease or a neurodegenerative disease. In a non-limiting embodiment, the length of a polypeptide comprising the Replikin peptide sequence or homologue may be one, five, ten, twenty, thirty, forty, fifty or more amino acid residues longer than the identified Replikin sequence with which it is homologous. Binding agents are also provided that bind to at least a portion of an amino acid sequence of at least one of SEQ ID NO(s): 1-8.

Binding agents may specifically or preferentially bind to the target protein, protein fragment, polypeptide, or peptide. Binding agents may specifically or preferentially bind to a homologue of at least one of SEQ ID NO(s): 1-8. Binding agents may likewise specifically or preferentially bind to a peptide sequence consisting of any one of SEQ ID NO(s): 1-8. Binding agents may also specifically or preferentially bind to a portion of a peptide consisting of any one of SEQ ID NO(s): 1-8 including a single amino acid within a homologue of SEQ ID NO(s): 1-8, two amino acids, three amino acids, four amino acids, five amino acids, or any number of amino acids spread within or outside a homologue.

In a non-limiting embodiment, the isolated Replikin peptide sequences may be used to generate antibodies, which may be used, for example, for diagnostic purposes, to identify protein or protein fragments of interest for development of vaccines and other therapies, or, for example, to provide passive immunity in an subject. Various procedures known in the art may be used for the production of antibodies to Replikin sequences. Such antibodies include but are not limited to polyclonal, monoclonal, chimeric, humanized, single chain, Fab fragments and fragments produced by a Fab expression library. Antibodies that are linked to a cytotoxic agent or signal may also be generated. Furthermore, combinations of antibodies to different Replikin sequences may be administered as an antibody cocktail.

An antibody of one aspect of the invention may bind to a Replikin peptide sequence or a Replikin Peak Gene sequence. It may bind to a protein or protein fragment comprising a Replikin peptide or a Replikin Peak Gene. It may also bind to a portion of a Replikin peptide or a portion of a Replikin Peak Gene or a portion of a protein, protein fragment, polypeptide, or peptide comprising a Replikin peptide or Replikin Peak Gene. A Replikin Peak Gene is an amino acid sequence having the highest concentration of continuous, non-interrupted, and/or overlapping Replikin sequences as compared to other sequences in a protein or genome.

An antibody that specifically binds to a portion of a Replikin peptide or a portion of a Replikin Peak Gene generally binds to an epitope on the Replikin peptide or an epitope that is at least partially on the Replikin peptide or to an epitope on the Replikin Peak Gene or an epitope that is at least partially on the Replikin Peak Gene when the antibody or fragment of the antibody binds to the epitope more readily than it would bind to a random, unrelated epitope.

Monoclonal antibodies to Replikin sequences may be prepared by using any technique that provides for the production of antibody molecules. These include but are not limited to the hybridoma technique originally described by Kohler and Milstein, (Nature, 1975, 256:495-497), the human B-cell hybridoma technique (Kosbor et al., 1983, Immunology Today, 4:72), and the EBV hybridoma technique (Cole et al., Monoclonal Antibodies and Cancer Therapy, Alan R. Liss, Inc., pp. 77-96). In addition, techniques developed for the production of chimeric antibodies (Morrison et al., 1984, Proc. Nat. Acad. Sci USA, 81:6851-6855) or other techniques may be used. Alternatively, techniques described for the production of single chain antibodies (U.S. Pat. No. 4,946,778) can be adapted to produce Replikin-specific single chain antibodies.

Antibodies to any peptides observed to be present in a prion disease or a neurodegenerative disease and combinations of such antibodies are useful in the treatment and/or prevention of a prion disease or a neurodegenerative disease, including Replikin peptide sequences and functional fragments thereof, Replikin Peak Gene peptide sequences, and Replikin sequences isolated within Replikin Peak Gene peptide sequences.

Antibody fragments that contain binding sites for a Replikin may be generated by known techniques. For example, such fragments include but are not limited to F(ab′) 2 fragments which can be produced by pepsin digestion of the antibody molecules and the Fab fragments that can be generated by reducing the disulfide bridges of the F(ab′)2 fragments. Alternatively, Fab expression libraries can be generated (Huse et al., 1989, Science, 246:1275-1281) to allow rapid and easy identification of monoclonal Fab fragments with the desired specificity.

In another aspect of the invention, immune serum containing antibodies to one or more Replikin sequences obtained from an individual exposed to one or more Replikin sequences may be used to induce passive immunity in another individual or animal. Immune serum may be administered via i.v. to a subject in need of treatment. Passive immunity also can be achieved by injecting a recipient with preformed antibodies to one or more Replikin sequences or functional fragments thereof. Passive immunization may be used to provide immediate protection to individuals who have been exposed to a prion. Administration of immune serum or preformed antibodies is routine and the skilled practitioner can readily ascertain the amount of serum or antibodies needed to achieve the desired effect. One of the reasons that vaccines directed towards a particular protein antigen of a disease-causing agent have not been fully effective in providing protection against the disease is that the best antibodies have not been produced, that is—it is likely that the antibodies to the Replikin sequences or functional fragment thereof have not been produced.

Anti-Sense Nucleic Acids and siRNA

One aspect of the invention further provides a nucleic acid sequence that is antisense to a nucleic acid sequence that encodes for any Replikin peptide present in or identified in a prion disease or a neurodegenerative disease. This may include one of SEQ ID NO(s): 1-8 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide sequence of identified in a prion disease or other neurodegenerative disease including, for example, any one of SEQ ID NO(s): 1-8 or is 30%, 40%, 50%, 60%, 70%, 80%, 90%, 95% or more homologous with a nucleic acid that is antisense to a nucleic acid that encodes for any one of SEQ ID NO(s): 1-8.

The nucleotide sequence of the invention may be used in hybridization assays of biopsied tissue or blood, e.g., Southern or Northern analysis, including in situ hybridization assays, to diagnose the presence of a particular Replikin sequence in a tissue sample or an environmental sample, for example. The present invention also provides kits containing antibodies specific for particular Replikin sequences or functional fragments thereof that are present in a particular prion or neurodegenerative disease, or containing nucleic acid molecules (sense or antisense) that hybridize specifically to a particular Replikin sequence, and optionally, various buffers and/or reagents needed for diagnosis.

Also within the scope of the invention are oligoribonucleotide sequences that include antisense RNA and DNA molecules and ribozymes that function to inhibit the translation of Replikin-containing mRNA. Both antisense RNA and DNA molecules and ribozymes may be prepared by any method known in the art. The antisense molecules can be incorporated into a wide variety of vectors for delivery to a subject. The skilled practitioner can readily determine the best route of delivery, although generally intravenous or intramuscular delivery is routine. The dosage amount is also readily ascertainable.

An aspect of the invention further provides antisense nucleic acid molecules that are complementary to a nucleic acid of the invention, wherein the antisense nucleic acid molecule is complementary to a nucleotide sequence encoding a peptide of the invention. In particular the nucleic acid sequence may be anti-sense to a nucleic acid sequence that has been demonstrated to be conserved across prion or neurodegenerative diseases or across time and/or which are present in a prion disease or neurodegenerative disease observed to have an increase in concentration of Replikin sequences.

An aspect of the invention also provides compositions comprising RNAi-inducing entities used to inhibit or reduce prion infection or replication including small interfering RNA, which is a class of about 10 to about 50 and often about 20 to about 25 nucleotide-long double-stranded RNA molecules. siRNA is involved in the RNA interference pathway, where it interferes with the expression of a specific gene. siRNAs also act in RNAi-related pathways, e.g., as an anti-prion mechanism.

An effective amount of an RNAi-inducing entity is delivered to a subject prior to or at the time of infection or at the time of diagnosis of disease. A dosage may be sufficient to reduce or delay one or more symptoms of a prion or other neurodegenerative disease. Compositions of the invention may comprise a single siRNA species targeted to a target transcript or may comprise a plurality of different siRNA species targeting one or more target transcripts.

The invention provides a small interfering nucleic acid sequence that is about 10 to about 50 nucleic acids in length and is 30%, 40%, 50%, 60%, 70%, 80%, 90%, or 95% or more homologous with a nucleic acid that encodes for any portion of a Replikin peptide including, for example, any portion of SEQ ID NO(s): 1-8 or is 30%, 40%, 50%, 60%, 70%, 80%, 90%, or 95% or more homologous with a nucleic acid that is antisense to a nucleic acid that encodes for any portion of a Replikin peptide, including, for example, a portion of one of SEQ ID NO(s): 1-8. In a further non-limiting embodiment, the nucleic acid sequence is about 15 to about 30 nucleic acids. In a further non-limiting embodiment, the nucleic acid sequence is about 20 to about 25 nucleic acids. In a further non-limiting embodiment, the nucleic acid sequence is about 21 nucleic acids.

### Therapeutic Formulations

A therapeutic formulation, including a vaccine, may be formulated with a pharmaceutically acceptable excipient, carrier, or adjuvant. One pharmaceutically-acceptable carrier or excipient is water. Excipients, carriers, or adjuvants may include, but are not limited to, excipients, carriers and adjuvants known to those of skill in the art now or hereafter.

The compositions of an aspect of the invention may be formulated for delivery by any available route including, but not limited to parenteral (e.g., intravenous), intradermal, subcutaneous, oral, nasal, bronchial, ophthalmic, transdermal (topical), transmucosal or any other routes. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for intranasal, intraocular, spray inhalation, parenteral (e.g., intravenous), intramuscular, intradermal, or subcutaneous application can include the following components: a sterile diluent such as water (for dermal, nasal, or ocular application, spraying, or injection), saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. Preparations may be enclosed in ampoules, disposable syringes or multiple dose vials made of glass or plastic.

Pharmaceutical compositions suitable for injectable use typically include sterile aqueous solutions (water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above.

Administration of the vaccine via any method may produce an immune response in the animal or human, it may further produce an antibody response in the animal or human. In a further non-limiting embodiment, the vaccine may produce a protective effect in the animal or human. For example, the vaccine of an aspect of the present invention may be administered to a rabbit, a chicken, a shrimp, a pig, a ferret, a human, or any animal capable of an immune response or blocking response.

### Replikin Concentration in Prion and Other Neurodegenerative Diseases

One non-limiting aspect of the present invention provides a method of determining the Replikin concentration of a protein, protein fragment, polypeptide, or peptide of a prion or other neurodegenerative disease comprising identifying Replikin sequences in a protein, protein fragment, polypeptide, or peptide of a prion or other neurodegenerative disease and determining the number of Replikin sequences per 100 amino acid residues.

Example 9 below provides non-limiting examples of the determination of Replikin concentration in all published accession numbers at PubMed.com identified by the applicants as related to prion and other neurodegenerative diseases as well as mean Replikin concentrations for all accession numbers in a given year, standard deviation from the mean, and statistical significance.

Example 10 below provides Table 12, which is a table of all accession numbers determined by the applicants to be derived from or associated with prion and other neurodegenerative diseases. Table 12 also provides Replikin concentration of the published amino acid sequence, year, source, serotype (if known), strain (if known), and definition of the published sequence as presented in the publication of the accession number at PubMed.com.

Examples provided herein are only exemplary. One of ordinary skill in the art will understand the scope of the invention is not limited by the examples and will understand many aspects of the invention that may be practiced in view of the examples provided herein based on common knowledge in the art.

### Example 1

### Conservation of HTVTTTTKGENFTETDIK (SEQ ID NO: 1)

HTVTTTTKGENFTETDIK (SEQ ID NO: 1) was discovered as conserved in prion diseases. Table 1 provides the year, accession number, and position in prion protein in which the peptide was discovered within a prion disease. Conservation of the quadruple repeat of threonine is one aspect of the invention for diagnostic, therapeutic, and preventive uses.

TABLE 1
Conservation of SEQ ID NO: 1 in prion disease

| Year | Accession Numbers and Position of Peptide |
|---|---|
| 2000 | 1DX1_A position 176, 1DX0_A position 176, 1DWZ_A position 69, |
| | 1DWY_A position 69 |
| 2004 | 1TQC_A position 64, 1TQB_A position 64, 1TPX_A position 77 |
| 2005 | ABC15790 position 187, ABC15786 position 198, ABC15785 |
| | position 190 |
| 2007 | ABT02039 position 187, ABT02034 position 190, ABT02035 |
| | position 198 |
| 2010 | 3079_B position 62 |

### Example 2

### Conservation of HGGGGWGQGGTHGQWNKPSKPKTNMK (SEQ ID NO: 2)

HGGGGWGQGGTHGQWNKPSKPKTNMK (SEQ ID NO: 2) was discovered as conserved in prion diseases. Table 2 provides the year, accession number, and position in prion protein in which the peptide was discovered within a prion disease. Conservation of the quadruple repeat of glycine and the repeat of glycines throughout the sequence are both aspects of the invention for diagnostic, therapeutic, and preventive uses.

TABLE 2
Conservation of SEQ ID NO: 2 in prion disease

| Year | Accession Numbers and Position of Peptide |
|---|---|
| 2000 | 1DX1_A position 74, 1DX0_A position 74 |
| 2005 | ABC15786 position 96 |
| 2007 | ABT02035 position 96 |

### Example 3

### Conservation of HSQWNKPSKPKTNMK (SEQ ID NO: 3)

HSQWNKPSKPKTNMK (SEQ ID NO: 3) was discovered as conserved in prion diseases. Table 3 provides the year, accession number, and position in prion protein in which the peptide was discovered within a prion disease.

TABLE 3
Conservation of SEQ ID NO: 3 in prion disease

| Year | Accession Numbers and Position of Peptide |
|---|---|
| 1986 | P04156 position 96. |
| 1999 | NP_000302 position 96. |
| 2000 | 1F07_A position 7, 1FKC_A position 7 |
| 2003 | NP_898902 position 96 |
| 2005 | NP_001009093 position 96, ABC15789 position 96, ABC15788 |
| | position 96, ABC15785 position 99 |
| 2007 | NP_001103676 position 96, ABT02038 position 96, ABT02037 |
| | position 96, ABT02034 position 99 |
| 2009 | 2K1D_A position 11 |
| 2010 | 2KUN_A position 7 |
| 2011 | 2LEJ_A position 12 |

### Example 4

### Conservation of KPSKPKTNMKH (SEQ ID NO: 4)

KPSKPKTNMKH (SEQ ID NO: 4) was discovered as conserved in prion diseases. Table 4 provides the year, accession number, and position in prion protein in which the peptide was discovered within a prion disease.

TABLE 4
Conservation of SEQ ID NO: 4 in prion disease

| Year | Accession Numbers and Position of Peptide |
|---|---|
| 1986 | P04156 position 101 |
| 1999 | NP_000302 position 101 |
| 2000 | 1DX1_A position 90, 1FO7_A position 12, 1FKC_A position 12, |
| | 1DX0_A position 90 |
| 2003 | NP_898902 position 101 |
| 2005 | NP_001009093 position 101, ABC15790 position 101, ABC15789 |
| | position 101, ABC15788 position 101, ABC15787 position 105, |
| | ABC15786 position 112, ABC15785 position 104 |
| 2007 | NP_001103676 position 101, ABT02039 position 101, ABT02038 |
| | position 101, ABT02037 position 101, ABT02034 position 104, |
| | ABT02036 position 105, ABT02035 position 112 |
| 2008 | ABR23643 position 101 |
| 2009 | 2K1D_A position 16 |
| 2010 | 2KUN_A position 12 |
| 2011 | 2LEJ_A position 17 |

### Example 5

### Conservation of KQHTVTTTTK (SEQ ID NO: 5)

KQHTVTTTTK (SEQ ID NO: 5) was discovered as conserved in prion diseases. Table 5 provides the year, accession number, and position in prion protein in which the peptide was discovered within a prion disease. Conservation of the quadruple repeat of threonine is an aspect of the invention for diagnostic, therapeutic, and preventive uses.

TABLE 5
Conservation of SEQ ID NO: 5 in prion disease

| Year | Accession Numbers and Position of Peptide |
|---|---|
| 1986 | P04156 position 185 |
| 1999 | NP_000302 position 185 |
| 2000 | NP_036763 position 185, 1E1W_A position 61, 1E1U_A position |
| | 61, 1E1S_A position 61, 1E1P_A position 61, 1E1J_A position 61, |
| | 1E1G_A position 61, 1FO7_A position 96, 1FKC_A position 96 |
| 2003 | NP_898902 position 185 |
| 2004 | 1TQC_A position 62, 1TQB_A position 62, 1TPX_A position 75 |
| 2005 | NP_001009093 position 185, ABC15792 position 157, ABC15791 |
| | position 184, ABC15790 position 185, ABC15789 position 185, |
| | ABC15788 position 185, ABC15787 position 189, ABC15785 |
| | position 188 |
| 2007 | 1AG2_A position 62, NP_001103676 position 185, ABT02041 |
| | position 157, ABT02040 position 184, ABT02039 position 185, |
| | ABT02038 position 185, ABT02037 position 185, ABT02034 |
| | position 188, ABT02036 position 189, 2K5O_A position 67, 2K56_A |
| | position 67, ABR23643 position 185 |
| 2008 | 2K5O_A position 67, 2K56_A position 67, ABR23643 position 185 |
| 2009 | 2K1D_A position 100 |
| 2010 | 2KUN_A position 96, 3O79_B position 60 |
| 2011 | 2LEJ_A position 101 |

### Example 6

### Conservation of KPKTNMKH (SEQ ID NO: 6)

KPKTNMKH (SEQ ID NO: 6) was discovered as conserved in prion diseases. Table 6 provides the year, accession number, and position in prion protein in which the peptide was discovered within a prion disease.

TABLE 6
Conservation of SEQ ID NO: 6 in prion disease

| Year | Accession Numbers and Position of Peptide |
|---|---|
| 1986 | P04156 position 104 |
| 1999 | NP_000302 position 104 |
| 2000 | 1DX1_A position 93, 1FO7_A position 15, 1FKC_A position 15, |
| | 1DX0_A position 93 |
| 2003 | NP_898902 position 104 |
| 2005 | NP_001009093 position 104, ABC15790 position 104, ABC15789 |
| | position 104, ABC15788 position 104, ABC15787 position 108, |
| | ABC15786 position 115, ABC15785 position 107 |
| 2007 | NP_001103676 position 104, ABT02039 position 104, ABT02038 |
| | position 104, ABT02037 position 104, ABT02034 position 107, |
| | ABT02036 position 108, ABT02035 position 115 |
| 2008 | ABR23643 position 104 |
| 2009 | 2K1D_A position 19 |
| 2010 | 2KUN_A position 15 |
| 2011 | 2LEJ_A position 20 |

### Example 7

### Conservation of HFFFAKLNCRLYRK (SEQ ID NO: 7)

HFFFAKLNCRLYRK (SEQ ID NO: 7) was discovered as conserved in prion diseases and particularly discovered as conserved in association with serpin polymerization. Table 7 provides the year, accession number, and position in prion protein in which the peptide was discovered within a prion disease. Conservation of the triple repeat of phenylalanine is an aspect of the invention for diagnostic, therapeutic, and preventive uses.

TABLE 7
Conservation of SEQ ID NO: 7 in prion disease

| Year | Accession Numbers and Position of Peptide |
|---|---|
| 2008 | 2ZNH_B position 107, 2ZNH_A position 107 |

### Example 8

### Conservation of KFDTISEKTSDQIH (SEQ ID NO: 8)

KFDTISEKTSDQIH (SEQ ID NO: 8) was discovered as conserved in prion diseases and particularly discovered as conserved in association with serpin polymerization. Table 8 provides the year, accession number, and position in prion protein in which the peptide was discovered within a prion disease.

TABLE 8
Conservation of SEQ ID NO: 8 in prion disease

| Year | Accession Numbers and Position of Peptide |
|---|---|
| 2008 | 2ZNH_B position 107, 2ZNH_A position 107 |

### Example 9

### Replikin Concentration in Prion and Other Neurodegenerative Diseases

The applicants reviewed proteins published at PubMed.com to determine the Replikin concentration of the Alzheimer's prion for each year in which amino acid sequences of the prion were published from 1986 to 2012. Table 9 below provides the number of published isolates for each year, the mean Replikin concentration for each year, standard deviation from the mean, and significance.

The applicants further reviewed proteins published at PubMed.com to determine the Replikin concentration of Alzheimer's prion diseases for each year in which amino acid sequences of were published from 1986 to 2012. Table 10 below provides the number of published isolates for each year, the mean Replikin concentration for each year, standard deviation from the mean, and significance.

The applicants further reviewed proteins published at PubMed.com to determine the Replikin concentration of proteins from all prion diseases for each year in which amino acid sequences of prion diseases were published from 1986 to 2012. Table 11 below provides the number of published isolates for each year, the mean Replikin concentration for each year, standard deviation from the mean, and significance.

TABLE 9
Replikin concentration in the Alzheimer's prion as
published in PubMed for years from 1986 through 2012.

| | | | Mean | | |
|---|---|---|---|---|---|
| | Accession | No. of | Replikin | | |
| Year | Numbers | Isolates | Count | S.D. | Significance |
| 1986 | P04156 14 | 1 | 5.5 | 0.0 | |
| 1999 | NP_015557 2 | 2 | 0.4 | 0.0 | low p < .001 |
| | NP_000012 2 | | | | |
| 2000 | NP_036541 3 | 2 | 5.1 | 4.8 | low p > .50, |
| | NP_065177 26 | | | | prev p < .30 |
| 2002 | Q9UKY0 3 | 1 | 1.7 | 0.0 | prev p < .40 |
| 2003 | NP_877497 30 | 3 | 3.2 | 1.5 | low p < .10, |
| | NP_775768 3 | | | | prev p < .20 |
| | Q10741 31 | | | | |
| 2006 | NP_001073153 26 | 2 | 8.8 | 0.4 | low p < .05, |
| | NP_001073152 26 | | | | prev |
| | | | | | p < .005 |
| 2008 | YP_005389467 5 | 4 | 1.8 | 0.1 | low |
| | AFC66769 5 | | | | p < .001, |
| | 2ZNH_B 8 2ZNH_A | | | | prev |
| | 8 | | | | p < .001 |
| 2010 | NP_001182598 43 | 1 | 5.6 | 0.0 | prev |
| | | | | | p < .001 |
| 2012 | YP_005412230 5 | 2 | 1.7 | 0.0 | low |
| | AFC68698 5 | | | | p < .001, |
| | | | | | prev |
| | | | | | p < .001 |

TABLE 10
Replikin concentration in Alzheimer's Prion diseases as
published in PubMed for years from 1986 through 2012.

| | | | Mean | | |
|---|---|---|---|---|---|
| | Accession | No. of | Replikin | | |
| Year | Numbers | Isolates | Count | S.D. | Significance |
| 1986 | P04156 14 | 1 | 5.5 | 0.0 | |
| 2000 | NP_036541 3 | 1 | 1.7 | 0.0 | |
| 2002 | Q9UKY0 3 | 1 | 1.7 | 0.0 | |
| 2003 | NP_877497 30 | 2 | 2.7 | 1.7 | low p < .20, |
| | NP_775768 3 | | | | prev p > .50 |

TABLE 11
Replikin concentration in all prion diseases discovered by
the applicants as published in PubMed for years from 1986 through
2012.

| | | | Mean | | |
|---|---|---|---|---|---|
| | Accession | No. of | Replikin | | |
| Year | Numbers | Isolates | Count | S.D. | Significance |
| 1986 | P04156 14 | 1 | 5.5 | 0.0 | |
| 1999 | NP_000302 14 | 1 | 5.5 | 0.0 | |
| 2000 | NP_036541 3 | 15 | 5.2 | 2.1 | low p < .40, |
| | NP_036763 13 | | | | prev p < .40 |
| | NP_071912 7 | | | | |
| | 1DX1_A 14 1E1W_A | | | | |
| | 5 1E1U_A 5 1E1S_A | | | | |
| | 5 1E1P_A 5 1E1J_A | | | | |
| | 5 1E1G_A 5 1FO7_A | | | | |
| | 13 1FKC_A 13 | | | | |
| | 1DX0_A 14 1DWZ_A | | | | |
| | 5 1DWY_A 5 | | | | |
| 2001 | NP_114468 29 | 2 | 4.5 | 3.7 | low p > .50, |
| | 1I17_A 2 | | | | prev p > .50 |
| 2002 | Q9UKY0 3 | 1 | 1.7 | 0.0 | prev p < .40 |
| 2003 | NP_898902 14 | 3 | 3.6 | 2.1 | low p < .20, |
| | NP_877497 30 | | | | prev p < .20 |
| | NP_775768 3 | | | | |
| 2004 | 1TQC_C 3 1TQC_A 4 | 7 | 3.0 | 1.6 | low |
| | 1TQB_C 3 1TQB_A 4 | | | | p < .005, |
| | 1TPX_C 3 1TPX_A 4 | | | | prev p > .50 |
| | NP_001003978 35 | | | | |
| 2005 | Q9H2A9 7 | 10 | 4.9 | 1.2 | low p < .10, |
| | NP_001009093 14 | | | | prev p < .01 |
| | ABC15792 13 | | | | |
| | ABC15791 13 | | | | |
| | ABC15790 13 | | | | |
| | ABC15789 14 | | | | |
| | ABC15788 14 | | | | |
| | ABC15787 13 | | | | |
| | ABC15786 13 | | | | |
| | ABC15785 13 | | | | |
| 2007 | 1AG2_A 4 | 10 | 5.2 | 0.5 | low p < .04, |
| | NP_001103676 14 | | | | prev p > .50 |
| | ABT02041 13 | | | | |
| | ABT02040 13 | | | | |
| | ABT02039 13 | | | | |
| | ABT02038 14 | | | | |
| | ABT02037 14 | | | | |
| | ABT02034 13 | | | | |
| | ABT02036 13 | | | | |
| | ABT02035 13 | | | | |
| 2008 | ACJ36231 97 | 7 | 3.3 | 1.4 | low |
| | ACD36979 28 | | | | p < .002, |
| | NP_001121368 7 | | | | prev |
| | NP_001121367 7 | | | | p < .002 |
| | 2K5O_A 4 2K56 _A 4 | | | | |
| | ABR23643 13 | | | | |
| 2009 | 2K1D_A 10 | 3 | 4.0 | 2.5 | low p < .30, |
| | ACQ13333 7 | | | | prev p > .50 |
| | ACQ13332 7 | | | | |
| 2010 | 2KUN_A 13 3O79_B | 2 | 6.3 | 3.5 | low p > .50, |
| | 4 | | | | prev p < .40 |
| 2011 | AEG75818 63 | 2 | 9.4 | 3.6 | low p < .30, |
| | 2LEJ_A 10 | | | | prev p < .40 |

### Example 10

### Replikin Concentration in Protein Isolates from Prion and Neurodegenerative Diseases

The applicants reviewed proteins published at PubMed.com to determine the Replikin concentration of all prion and neurodegenerative diseases for each year in which amino acid sequences were published from 1986 to 2012. Table 12 below provides a listing of each accession number revealed in the review of the PubMed database along with Replikin concentration of the published amino acid sequence, year, source, serotype (if known), strain (if known), and definition of the published sequence as presented in the publication of the accession number at PubMed.com.

TABLE 12
Replikin concentration in proteins isolated from prion and other neurodegenerative diseases

| | REPLIKIN | | | | | |
|---|---|---|---|---|---|---|
| ACCESSION | CONCENTRATION | YEAR | SOURCE | SEROTYPE | STRAIN | DEFINITION |
| NP_775768 | 1.4 | 2003 | Homo sapiens | unknown | human | HECTD2 is associated with susceptibility to mouse |
| | | | (human) | | | and human prion disease Absence of association |
| | | | | | | between two HECTD2 polymorphisms and |
| | | | | | | sporadic Creutzfeldt-Jakob disease The DNA |
| | | | | | | sequence and comparative analysis of human |
| | | | | | | chromosome 10 A scan of chromosome 10 |
| | | | | | | identifies a novel locus showing strong association |
| | | | | | | with late-onset Alzheimer disease HECTD2; a |
| | | | | | | candidate susceptibility gene for Alzheimer's |
| | | | | | | disease on 10q |
| 1TPX_C | 1.4 | 2004 | Mus musculus | unknown | house | Direct Submission Insight into the PrPC-->PrPSc |
| | | | (house mouse) | | mouse | conversion from the structures of antibody-bound |
| | | | | | | ovine prion scrapie-susceptibility variants |
| 1TQB_C | 1.4 | 2004 | Mus musculus | unknown | house | Direct Submission Insight into the PrPC-->PrPSc |
| | | | (house mouse) | | mouse | conversion from the structures of antibody-bound |
| | | | | | | ovine prion scrapie-susceptibility variants |
| 1TQC_C | 1.4 | 2004 | Mus musculus | unknown | house | Direct Submission Insight into the PrPC-->PrPSc |
| | | | (house mouse) | | mouse | conversion from the structures of antibody-bound |
| | | | | | | ovine prion scrapie-susceptibility variants |
| NP_036541 | 1.7 | 2000 | Homo sapiens | unknown | human | Polymorphisms within the prion-like protein gene |
| | | | (human) | | | (Prnd) and their implications in human prion |
| | | | | | | diseases; Alzheimer's disease and other |
| | | | | | | neurological disorders Biochemical signatures of |
| | | | | | | doppel protein in human astrocytomas to support |
| | | | | | | prediction in tumor malignancy Doppel-induced |
| | | | | | | cerebellar degeneration in transgenic mice |
| | | | | | | Transient expressions of doppel and its structural |
| | | | | | | analog prionDelta32-121 in SH-SY5Y cells caused |
| | | | | | | cytotoxicity possibly by triggering similar |
| | | | | | | apoptosis pathway The DNA sequence and |
| | | | | | | comparative analysis of human chromosome 20 |
| | | | | | | The doppel (Dpl) protein influences in vitro |
| | | | | | | migration capability in astrocytoma-derived cells |
| | | | | | | First report of polymorphisms in the prion-like |
| | | | | | | protein gene (PRND): implications for human |
| | | | | | | prion diseases Earlier onset of Alzheimer's |
| | | | | | | disease: risk polymorphisms within PRNP; PRND; |
| | | | | | | CYP46; and APOE genes Expression and structural |
| | | | | | | characterization of the recombinant human |
| | | | | | | doppel protein Expression and structural |
| | | | | | | characterization of the recombinant human |
| | | | | | | doppel protein Doppel-induced cytotoxicity in |
| | | | | | | human neuronal SH-SY5Y cells is antagonized by |
| | | | | | | the prion protein |
| NP_071912 | 1.7 | 2000 | Homo sapiens | unknown | human | Molecular cloning and expression of the pituitary |
| | | | (human) | | | glycoprotein hormone N-acetylgalactosamine-4- |
| | | | | | | O-sulfotransferase Analyses of shared genetic |
| | | | | | | factors between asthma and obesity in children |
| | | | | | | Molecular cloning and characterization of GalNAc |
| | | | | | | 4-sulfotransferase expressed in human pituitary |
| | | | | | | gland Genome-wide pharmacogenomic study of |
| | | | | | | neurocognition as an indicator of antipsychotic |
| | | | | | | treatment response in schizophrenia Molecular |
| | | | | | | cloning and expression of two distinct human N- |
| | | | | | | acetylgalactosamine 4-O-sulfotransferases that |
| | | | | | | transfer sulfate to GalNAc beta 1-->4GlcNAc beta |
| | | | | | | 1-->R in both N- and O-glycans Glycosylation- |
| | | | | | | related gene expression in prion diseases: PrPSc |
| | | | | | | accumulation in scrapie infected GT1 cells |
| | | | | | | depends on beta-1; 4-linked GalNAc-4-SO4 |
| | | | | | | hyposulfation Differential expression and |
| | | | | | | enzymatic properties of GalNAc-4- |
| | | | | | | sulfotransferase-1 and GalNAc-4-sulfotransferase- |
| | | | | | | 2 Genome-wide association yields new sequence |
| | | | | | | variants at seven loci that associate with |
| | | | | | | measures of obesity |
| Q9UKY0 | 1.7 | 2002 | Homo sapiens | unknown | human | NMR structure of the human doppel protein |
| | | | (human) | | | Direct Submission Expression and structural |
| | | | | | | characterization of the recombinant human |
| | | | | | | doppel protein Ataxia in prion protein (PrP)- |
| | | | | | | deficient mice is associated with upregulation of |
| | | | | | | the novel PrP-like protein doppel The status; |
| | | | | | | quality; and expansion of the NIH full-length cDNA |
| | | | | | | project: the Mammalian Gene Collection (MGC) |
| | | | | | | The DNA sequence and comparative analysis of |
| | | | | | | human chromosome 20 The secreted protein |
| | | | | | | discovery initiative (SPDI); a large-scale effort to |
| | | | | | | identify novel human secreted and |
| | | | | | | transmembrane proteins: a bioinformatics |
| | | | | | | assessment First report of polymorphisms in the |
| | | | | | | prion-like protein gene (PRND): implications for |
| | | | | | | human prion diseases Polymorphisms within the |
| | | | | | | prion-like protein gene (Prnd) and their |
| | | | | | | implications in human prion diseases; Alzheimer's |
| | | | | | | disease and other neurological disorders Direct |
| | | | | | | Submission |
| Q9H2A9 | 1.7 | 2005 | Homo sapiens | unknown | human | Molecular cloning and characterization of GalNAc |
| | | | (human) | | | 4-sulfotransferase expressed in human pituitary |
| | | | | | | gland Molecular cloning and expression of the |
| | | | | | | pituitary glycoprotein hormone N- |
| | | | | | | acetylgalactosamine-4-O-sulfotransferase The |
| | | | | | | consensus coding sequences of human breast and |
| | | | | | | colorectal cancers Glycosylation-related gene |
| | | | | | | expression in prion diseases: PrPSc accumulation |
| | | | | | | in scrapie infected GT1 cells depends on beta-1; 4- |
| | | | | | | linked GalNAc-4-SO4 hyposulfation The status; |
| | | | | | | quality; and expansion of the NIH full-length cDNA |
| | | | | | | project: the Mammalian Gene Collection (MGC) |
| | | | | | | Molecular cloning and expression of two distinct |
| | | | | | | human N-acetylgalactosamine 4-O- |
| | | | | | | sulfotransferases that transfer sulfate to GalNAc |
| | | | | | | beta 1-->4GlcNAc beta 1-->R in both N- and O- |
| | | | | | | glycans |
| NP_001121367 | 1.7 | 2008 | Homo sapiens | unknown | human | Molecular cloning and expression of the pituitary |
| | | | (human) | | | glycoprotein hormone N-acetylgalactosamine-4- |
| | | | | | | O-sulfotransferase Analyses of shared genetic |
| | | | | | | factors between asthma and obesity in children |
| | | | | | | Molecular cloning and characterization of GalNAc |
| | | | | | | 4-sulfotransferase expressed in human pituitary |
| | | | | | | gland Genome-wide pharmacogenomic study of |
| | | | | | | neurocognition as an indicator of antipsychotic |
| | | | | | | treatment response in schizophrenia Molecular |
| | | | | | | cloning and expression of two distinct human N- |
| | | | | | | acetylgalactosamine 4-O-sulfotransferases that |
| | | | | | | transfer sulfate to GalNAc beta 1-->4GlcNAc beta |
| | | | | | | 1-->R in both N- and O-glycans Glycosylation- |
| | | | | | | related gene expression in prion diseases: PrPSc |
| | | | | | | accumulation in scrapie infected GT1 cells |
| | | | | | | depends on beta-1; 4-linked GalNAc-4-SO4 |
| | | | | | | hyposulfation Differential expression and |
| | | | | | | enzymatic properties of GalNAc-4- |
| | | | | | | sulfotransferase-1 and GalNAc-4-sulfotransferase- |
| | | | | | | 2 Genome-wide association yields new sequence |
| | | | | | | variants at seven loci that associate with |
| | | | | | | measures of obesity |
| NP_001121368 | 1.7 | 2008 | Homo sapiens | unknown | human | Molecular cloning and expression of the pituitary |
| | | | (human) | | | glycoprotein hormone N-acetylgalactosamine-4- |
| | | | | | | O-sulfotransferase Analyses of shared genetic |
| | | | | | | factors between asthma and obesity in children |
| | | | | | | Molecular cloning and characterization of GalNAc |
| | | | | | | 4-sulfotransferase expressed in human pituitary |
| | | | | | | gland Genome-wide pharmacogenomic study of |
| | | | | | | neurocognition as an indicator of antipsychotic |
| | | | | | | treatment response in schizophrenia Molecular |
| | | | | | | cloning and expression of two distinct human N- |
| | | | | | | acetylgalactosamine 4-O-sulfotransferases that |
| | | | | | | transfer sulfate to GalNAc beta 1-->4GlcNAc beta |
| | | | | | | 1-->R in both N- and O-glycans Glycosylation- |
| | | | | | | related gene expression in prion diseases: PrPSc |
| | | | | | | accumulation in scrapie infected GT1 cells |
| | | | | | | depends on beta-1; 4-linked GalNAc-4-SO4 |
| | | | | | | hyposulfation Differential expression and |
| | | | | | | enzymatic properties of GalNAc-4- |
| | | | | | | sulfotransferase-1 and GalNAc-4-sulfotransferase- |
| | | | | | | 2 Genome-wide association yields new sequence |
| | | | | | | variants at seven loci that associate with |
| | | | | | | measures of obesity |
| 1I17_A | 1.9 | 2001 | Mus musculus | unknown | house | Ataxia in prion protein (PrP)-deficient mice is |
| | | | (house mouse) | | mouse | associated with upregulation of the novel PrP-like |
| | | | | | | protein doppel Two different neurodegenerative |
| | | | | | | diseases caused by proteins with similar |
| | | | | | | structures Direct Submission |
| ACQ13333 | 2.5 | 2009 | Unknown. | unknown | unknown | Single-chain antibody acting against 37 kDa/67 |
| | | | | | | kDa laminin receptor as tools for the diagnosis |
| | | | | | | and therapy of prion diseases and cancer; |
| | | | | | | production and use thereof |
| ACQ13332 | 2.6 | 2009 | Unknown. | unknown | unknown | Single-chain antibody acting against 37 kDa/67 |
| | | | | | | kDa laminin receptor as tools for the diagnosis |
| | | | | | | and therapy of prion diseases and cancer; |
| | | | | | | production and use thereof |
| ACJ36231 | 2.8 | 2008 | West Nile virus | unknown | NY99- | A PCR-based protocol for the generation of |
| | | | (WNV) | | 6922 | recombinant West Nile virus Development and |
| | | | | | | application of West Nile virus subgenomic |
| | | | | | | replicon RNA expressing secreted alkaline |
| | | | | | | phosphatase Direct Submission |
| 1TPX_A | 3.3 | 2004 | Ovis aries | unknown | sheep | Insight into the PrPC-->PrPSc conversion from the |
| | | | (sheep) | | | structures of antibody-bound ovine prion scrapie- |
| | | | | | | susceptibility variants Direct Submission |
| 2K56_A | 3.5 | 2008 | Myodes | unknown | Bank vole | NMR structure of the bank vole prion protein at |
| | | | glareolus | | | 20 degrees C. contains a structured loop of |
| | | | (Bank vole) | | | residues 165-171 Direct Submission |
| 2K5O_A | 3.5 | 2008 | Mus musculus | unknown | house | NMR structure of the bank vole prion protein at |
| | | | (house mouse) | | mouse | 20 degrees C. contains a structured loop of |
| | | | | | | residues 165-171 Direct Submission |
| 3O79_B | 3.8 | 2010 | Oryctolagus | unknown | rabbit | Prion disease susceptibility is affected by beta- |
| | | | cuniculus | | | structure folding propensity and local side-chain |
| | | | (rabbit) | | | interactions in PrP Direct Submission |
| NP_877497 | 3.9 | 2003 | Homo sapiens | unknown | human | HECTD2 is associated with susceptibility to mouse |
| | | | (human) | | | and human prion disease Absence of association |
| | | | | | | between two HECTD2 polymorphisms and |
| | | | | | | sporadic Creutzfeldt-Jakob disease The DNA |
| | | | | | | sequence and comparative analysis of human |
| | | | | | | chromosome 10 A scan of chromosome 10 |
| | | | | | | identifies a novel locus showing strong association |
| | | | | | | with late-onset Alzheimer disease HECTD2; a |
| | | | | | | candidate susceptibility gene for Alzheimer's |
| | | | | | | disease on 10q |
| 1TQB_A | 3.9 | 2004 | Ovis aries | unknown | sheep | Insight into the PrPC-->PrPSc conversion from the |
| | | | (sheep) | | | structures of antibody-bound ovine prion scrapie- |
| | | | | | | susceptibility variants Direct Submission |
| 1TQC_A | 3.9 | 2004 | Ovis aries | unknown | sheep | Insight into the PrPC-->PrPSc conversion from the |
| | | | (sheep) | | | structures of antibody-bound ovine prion scrapie- |
| | | | | | | susceptibility variants Direct Submission |
| 1AG2_A | 3.9 | 2007 | Mus musculus | unknown | house | NMR structure of the mouse prion protein domain |
| | | | (house mouse) | | mouse | PrP(121-321) Direct Submission |
| ACD36979 | 4.4 | 2008 | Mesocricetus | unknown | Golden | Direct Submission Differential expression of |
| | | | auratus | | Syrian | interferon responsive genes in rodent models of |
| | | | (golden | | | transmissible spongiform encephalopathy disease |
| | | | hamster) | | | |
| 1DWY_A | 4.5 | 2000 | Bos taurus | unknown | cattle | NMR structure of the bovine prion protein Direct |
| | | | (cattle) | | | Submission |
| 1DWZ_A | 4.5 | 2000 | Bos taurus | unknown | cattle | NMR structure of the bovine prion protein Direct |
| | | | (cattle) | | | Submission |
| 1E1G_A | 4.8 | 2000 | Homo sapiens | unknown | human | NMR structures of three single-residue variants of |
| | | | (human) | | | the human prion protein Direct Submission |
| 1E1J_A | 4.8 | 2000 | Homo sapiens | unknown | human | NMR structures of three single-residue variants of |
| | | | (human) | | | the human prion protein Direct Submission |
| 1E1P_A | 4.8 | 2000 | Homo sapiens | unknown | human | NMR structures of three single-residue variants of |
| | | | (human) | | | the human prion protein Direct Submission |
| 1E1S_A | 4.8 | 2000 | Homo sapiens | unknown | human | NMR structures of three single-residue variants of |
| | | | (human) | | | the human prion protein Direct Submission |
| 1E1U_A | 4.8 | 2000 | Homo sapiens | unknown | human | NMR structures of three single-residue variants of |
| | | | (human) | | | the human prion protein Direct Submission |
| 1E1W_A | 4.8 | 2000 | Homo sapiens | unknown | human | NMR structures of three single-residue variants of |
| | | | (human) | | | the human prion protein Direct Submission |
| ABC15786 | 4.9 | 2005 | Unknown. | unknown | unknown | Method for the detection of prion diseases |
| ABT02035 | 4.9 | 2007 | Unknown. | unknown | unknown | Method for the detection of prion diseases |
| NP_036763 | 5.1 | 2000 | Rattus | unknown | unknown | Prion protein (PrP) is not involved in the |
| | | | norvegicus | | | pathogenesis of spongiform encephalopathy in |
| | | | (Norway rat) | | | zitter rats The octapeptide repeat PrP(C) region |
| | | | | | | and cobalamin-deficient polyneuropathy of the |
| | | | | | | rat Three-exon structure of the gene encoding the |
| | | | | | | rat prion protein and its expression in tissues |
| | | | | | | Cobalamin (vitamin B(12)) regulation of PrP(C); |
| | | | | | | PrP(C)-mRNA and copper levels in rat central |
| | | | | | | nervous system Identification of a promoter |
| | | | | | | region in the rat prion protein gene The alpha- |
| | | | | | | secretase-derived N-terminal product of cellular |
| | | | | | | prion; N1; displays neuroprotective function in |
| | | | | | | vitro and in vivo Expression of mutant or cytosolic |
| | | | | | | PrP in transgenic mice and cells is not associated |
| | | | | | | 0with endoplasmic reticulum stress or proteasome |
| | | | | | | dysfunction Creutzfeldt-Jacob disease associated |
| | | | | | | with the PRNP codon 200Lys mutation: an analysis |
| | | | | | | of 45 families Cloning of rat ‘prion-related protein’ |
| | | | | | | cDNA Cellular prion protein localizes to the |
| | | | | | | nucleus of endocrine and neuronal cells and |
| | | | | | | interacts with structural chromatin components |
| ABC15785 | 5.1 | 2005 | Unknown. | unknown | unknown | Method for the detection of prion diseases |
| ABC15787 | 5.1 | 2005 | Unknown. | unknown | unknown | Method for the detection of prion diseases |
| ABC15790 | 5.1 | 2005 | Unknown. | unknown | unknown | Method for the detection of prion diseases |
| ABC15791 | 5.1 | 2005 | Unknown. | unknown | unknown | Method for the detection of prion diseases |
| ABT02034 | 5.1 | 2007 | Unknown. | unknown | unknown | Method for the detection of prion diseases |
| ABT02036 | 5.1 | 2007 | Unknown. | unknown | unknown | Method for the detection of prion diseases |
| ABT02039 | 5.1 | 2007 | Unknown. | unknown | unknown | Method for the detection of prion diseases |
| ABT02040 | 5.1 | 2007 | Unknown. | unknown | unknown | Method for the detection of prion diseases |
| ABR23643 | 5.3 | 2008 | Acomys | unknown | Egyptian | Prion protein amino acid determinants of |
| | | | cahirinus | | spiny | differential susceptibility and molecular feature of |
| | | | (Egyptian spiny | | mouse | prion strains in mice and voles Susceptibility of |
| | | | mouse) | | | Acomys cahirinus to prion diseases Direct |
| | | | | | | Submission |
| P04156 | 5.5 | 1986 | Homo sapiens | unknown | human | Mutant prion proteins in Gerstmann-Straussler- |
| | | | (human) | | | Scheinker disease with neurofibrillary tangles |
| | | | | | | Prion fibrillization is mediated by a native |
| | | | | | | structural element that comprises helices H2 and |
| | | | | | | H3 Early onset prion disease from octarepeat |
| | | | | | | expansion correlates with copper binding |
| | | | | | | properties Phenotypic variability of Gerstmann- |
| | | | | | | Straussler-Scheinker disease is associated with |
| | | | | | | prion protein heterogeneity Molecular genetics of |
| | | | | | | human prion diseases in Germany Crystal |
| | | | | | | structure of human prion protein bound to a |
| | | | | | | therapeutic antibody Direct Submission SSCP |
| | | | | | | analysis and sequencing of the human prion |
| | | | | | | protein gene (PRNP) detects two different 24 bp |
| | | | | | | deletions in an atypical Alzheimer's disease family |
| | | | | | | A variant of Gerstmann-Straussler-Scheinker |
| | | | | | | disease carrying codon 105 mutation with codon |
| | | | | | | 129 polymorphism of the prion protein gene: a |
| | | | | | | clinicopathological study NMR solution structure |
| | | | | | | of the human prion protein A new PRNP mutation |
| | | | | | | (G131V) associated with Gerstmann-Straussler- |
| | | | | | | Scheinker disease Pro----leu change at position |
| | | | | | | 102 of prion protein is the most common but not |
| | | | | | | the sole mutation related to Gerstmann-Straussler |
| | | | | | | syndrome Human prion protein cDNA: molecular |
| | | | | | | cloning; chromosomal mapping; and biological |
| | | | | | | implications The octarepeat domain of the prion |
| | | | | | | protein binds Cu(II) with three distinct |
| | | | | | | coordination modes at pH 7.4 Gerstmann- |
| | | | | | | Straussler-Scheinker disease with mutation at |
| | | | | | | codon 102 and methionine at codon 129 of PRNP |
| | | | | | | in previously unreported patients Molecular |
| | | | | | | features of the copper binding sites in the |
| | | | | | | octarepeat domain of the prion protein Genomic |
| | | | | | | structure of the human prion protein gene |
| | | | | | | Mutation in codon 200 of scrapie amyloid protein |
| | | | | | | gene in two clusters of Creutzfeldt-Jakob disease |
| | | | | | | in Slovakia Mass-spectrometric identification and |
| | | | | | | relative quantification of N-linked cell surface |
| | | | | | | glycoproteins Familial spongiform encephalopathy |
| | | | | | | associated with a novel prion protein gene |
| | | | | | | mutation Atomic structures of amyloid cross-beta |
| | | | | | | spines reveal varied steric zippers Genetic and |
| | | | | | | infectious prion diseases Direct Submission A |
| | | | | | | prion-linked psychiatric disorder A missense |
| | | | | | | mutation at codon 105 with codon 129 |
| | | | | | | polymorphism of the prion protein gene in a new |
| | | | | | | variant of Gerstmann-Straussler-Scheinker disease |
| | | | | | | Solution structure of the E200K variant of human |
| | | | | | | prion protein. Implications for the mechanism of |
| | | | | | | pathogenesis in familial prion diseases |
| | | | | | | Identification of three novel mutations (E196K; |
| | | | | | | V203I; E211Q) in the prion protein gene (PRNP) in |
| | | | | | | inherited prion diseases with Creutzfeldt-Jakob |
| | | | | | | disease phenotype Linkage of a prion protein |
| | | | | | | missense variant to Gerstmann-Straussler |
| | | | | | | syndrome The status; quality; and expansion of |
| | | | | | | the NIH full-length cDNA project: the Mammalian |
| | | | | | | Gene Collection (MGC) Transmissible familial |
| | | | | | | Creutzfeldt-Jakob disease associated with five; |
| | | | | | | seven; and eight extra octapeptide coding repeats |
| | | | | | | in the PRNP gene Mutation in codon 200 and |
| | | | | | | polymorphism in codon 129 of the prion protein |
| | | | | | | gene in Libyan Jews with Creutzfeldt-Jakob |
| | | | | | | disease Crystal structure of the human prion |
| | | | | | | protein reveals a mechanism for oligomerization |
| | | | | | | Molecular cloning of a human prion protein cDNA |
| | | | | | | New mutation in scrapie amyloid precursor gene |
| | | | | | | (at codon 178) in Finnish Creutzfeldt-Jakob |
| | | | | | | kindred Biosynthesis of prion protein |
| | | | | | | nucleocytoplasmic isoforms by alternative |
| | | | | | | initiation of translation Mutation of the prion |
| | | | | | | protein gene at codon 208 in familial Creutzfeldt- |
| | | | | | | Jakob disease Mutations and polymorphisms in |
| | | | | | | the prion protein gene Novel PRNP sequence |
| | | | | | | variant associated with familial encephalopathy |
| | | | | | | Deletion in the prion protein gene in a demented |
| | | | | | | patient Novel missense variants of prion protein |
| | | | | | | in Creutzfeldt-Jakob disease or Gerstmann- |
| | | | | | | Straussler syndrome A new point mutation of the |
| | | | | | | prion protein gene in Creutzfeldt-Jakob disease |
| | | | | | | Copper (II) promotes the formation of soluble |
| | | | | | | neurotoxic PrP oligomers in acidic environment |
| | | | | | | High prevalence of pathogenic mutations in |
| | | | | | | patients with early-onset dementia detected by |
| | | | | | | sequence analyses of four different genes |
| | | | | | | Conformational diversity in prion protein variants |
| | | | | | | influences intermolecular beta-sheet formation |
| | | | | | | The DNA sequence and comparative analysis of |
| | | | | | | human chromosome 20 Amyloid protein of |
| | | | | | | Gerstmann-Straussler-Scheinker disease (Indiana |
| | | | | | | kindred) is an 11 kd fragment of prion protein |
| | | | | | | with an N-terminal glycine at codon 58 Japanese |
| | | | | | | family with Creutzfeldt-Jakob disease with codon |
| | | | | | | 200 point mutation of the prion protein gene |
| | | | | | | NMR structures of three single-residue variants of |
| | | | | | | the human prion protein Fatal familial insomnia: a |
| | | | | | | second kindred with mutation of prion protein |
| | | | | | | gene at codon 178 The prion protein is a |
| | | | | | | combined zinc and copper binding protein: Zn2+ |
| | | | | | | alters the distribution of Cu2+ coordination |
| | | | | | | modes Polymorphism at codon 129 or codon 219 |
| | | | | | | of PRNP and clinical heterogeneity in a previously |
| | | | | | | unreported family with Gerstmann-Straussler- |
| | | | | | | Scheinker disease (PrP-P102L mutation) The |
| | | | | | | octapeptide repeats in mammalian prion protein |
| | | | | | | constitute a pH-dependent folding and |
| | | | | | | aggregation site Complete genomic sequence and |
| | | | | | | analysis of the prion protein gene region from |
| | | | | | | three mammalian species |
| NP_000302 | 5.5 | 1999 | Homo sapiens | unknown | human | Fatal familial insomnia; a prion disease with a |
| | | | (human) | | | mutation at codon 178 of the prion protein gene |
| | | | | | | Cleavage of the amino terminus of the prion |
| | | | | | | protein by reactive oxygen species alpha- |
| | | | | | | Secretase-derived fragment of cellular prion; N1; |
| | | | | | | protects against monomeric and oligomeric |
| | | | | | | amyloid beta (Abeta)-associated cell death Fatal |
| | | | | | | familial insomnia: a second kindred with mutation |
| | | | | | | of prion protein gene at codon 178 Recombinant |
| | | | | | | human prion protein mutants huPrP D178N/M129 |
| | | | | | | (FFI) and huPrP + 9OR (fCJD) reveal proteinase K |
| | | | | | | resistance Intraneuronal immunoreactivity for the |
| | | | | | | prion protein distinguishes a subset of E200K |
| | | | | | | genetic from sporadic Creutzfeldt-Jakob Disease |
| | | | | | | Mutant prion proteins in Gerstmann-Straussler- |
| | | | | | | Scheinker disease with neurofibrillary tangles |
| | | | | | | Alpha- and beta- cleavages of the amino-terminus |
| | | | | | | of the cellular prion protein Inherited prion |
| | | | | | | disease with 144 base pair gene insertion. 1. |
| | | | | | | Genealogical and molecular studies Gerstmann- |
| | | | | | | Straussler-Scheinker disease Accumulation of |
| | | | | | | transcripts coding for prion protein in human |
| | | | | | | astrocytes during infection with human |
| | | | | | | immunodeficiency virus Interactions between the |
| | | | | | | conserved hydrophobic region of the prion |
| | | | | | | protein and dodecylphosphocholine micelles Post- |
| | | | | | | translational hydroxylation at the N-terminus of |
| | | | | | | the prion protein reveals presence of PPII |
| | | | | | | structure in vivo Truncated forms of the human |
| | | | | | | prion protein in normal brain and in prion |
| | | | | | | diseases Influence of the pathogenic mutations |
| | | | | | | T188K/R/A on the structural stability and |
| | | | | | | misfolding of human prion protein: insight from |
| | | | | | | molecular dynamics simulations |
| NP_898902 | 5.5 | 2003 | Homo sapiens | unknown | human | Fatal familial insomnia; a prion disease with a |
| | | | (human) | | | mutation at codon 178 of the prion protein gene |
| | | | | | | Cleavage of the amino terminus of the prion |
| | | | | | | protein by reactive oxygen species alpha- |
| | | | | | | Secretase-derived fragment of cellular prion; N1; |
| | | | | | | protects against monomeric and oligomeric |
| | | | | | | amyloid beta (Abeta)-associated cell death Fatal |
| | | | | | | familial insomnia: a second kindred with mutation |
| | | | | | | of prion protein gene at codon 178 Recombinant |
| | | | | | | human prion protein mutants huPrP D178N/M129 |
| | | | | | | (FFI) and huPrP + 9OR (fCJD) reveal proteinase K |
| | | | | | | resistance Intraneuronal immunoreactivity for the |
| | | | | | | prion protein distinguishes a subset of E200K |
| | | | | | | genetic from sporadic Creutzfeldt-Jakob Disease |
| | | | | | | Mutant prion proteins in Gerstmann-Straussler- |
| | | | | | | Scheinker disease with neurofibrillary tangles |
| | | | | | | Alpha- and beta- cleavages of the amino-terminus |
| | | | | | | of the cellular prion protein Inherited prion |
| | | | | | | disease with 144 base pair gene insertion. 1. |
| | | | | | | Genealogical and molecular studies Gerstmann- |
| | | | | | | Straussler-Scheinker disease Accumulation of |
| | | | | | | transcripts coding for prion protein in human |
| | | | | | | astrocytes during infection with human |
| | | | | | | immunodeficiency virus Interactions between the |
| | | | | | | conserved hydrophobic region of the prion |
| | | | | | | protein and dodecylphosphocholine micelles Post- |
| | | | | | | translational hydroxylation at the N-terminus of |
| | | | | | | the prion protein reveals presence of PPII |
| | | | | | | structure in vivo Truncated forms of the human |
| | | | | | | prion protein in normal brain and in prion |
| | | | | | | diseases Influence of the pathogenic mutations |
| | | | | | | T188K/R/A on the structural stability and |
| | | | | | | misfolding of human prion protein: insight from |
| | | | | | | molecular dynamics simulations |
| NP_001003978 | 5.5 | 2004 | Rattus | unknown | Norway | Molecular cloning of a novel member of the |
| | | | norvegicus | | rat | eukaryotic polypeptide chain-releasing factors |
| | | | (Norway rat) | | | (eRF). Its identification as eRF3 interacting with |
| | | | | | | eRF1 The polypeptide chain-releasing factor |
| | | | | | | GSPT1/eRF3 is proteolytically processed into an |
| | | | | | | IAP-binding protein [Human transmissible |
| | | | | | | dementia: prion diseases?] |
| NP_001009093 | 5.5 | 2005 | Pan | unknown | chimpanzee | Prion protein gene variation among primates |
| | | | troglodytes | | | Mapping of chimpanzee full-length cDNAs onto |
| | | | (chimpanzee) | | | the human genome unveils large potential |
| | | | | | | divergence of the transcriptome Infectious |
| | | | | | | amyloid precursor gene sequences in primates |
| | | | | | | used for experimental transmission of human |
| | | | | | | spongiform encephalopathy Accelerated |
| | | | | | | evolution of nervous system genes in the origin of |
| | | | | | | Homo sapiens Variation of the prion gene in |
| | | | | | | chimpanzees and its implication for prion diseases |
| ABC15788 | 5.5 | 2005 | Unknown. | unknown | unknown | Method for the detection of prion diseases |
| ABC15789 | 5.5 | 2005 | Unknown. | unknown | unknown | Method for the detection of prion diseases |
| NP_001103676 | 5.5 | 2007 | Pan | unknown | chimpanzee | Prion protein gene variation among primates |
| | | | troglodytes | | | Mapping of chimpanzee full-length cDNAs onto |
| | | | (chimpanzee) | | | the human genome unveils large potential |
| | | | | | | divergence of the transcriptome Infectious |
| | | | | | | amyloid precursor gene sequences in primates |
| | | | | | | used for experimental transmission of human |
| | | | | | | spongiform encephalopathy Accelerated |
| | | | | | | evolution of nervous system genes in the origin of |
| | | | | | | Homo sapiens Variation of the prion gene in |
| | | | | | | chimpanzees and its implication for prion diseases |
| ABT02037 | 5.5 | 2007 | Unknown. | unknown | unknown | Method for the detection of prion diseases |
| ABT02038 | 5.5 | 2007 | Unknown. | unknown | unknown | Method for the detection of prion diseases |
| ABC15792 | 5.8 | 2005 | Unknown. | unknown | unknown | Method for the detection of prion diseases |
| ABT02041 | 5.8 | 2007 | Unknown. | unknown | unknown | Method for the detection of prion diseases |
| 1DX0_A | 6.4 | 2000 | Bos taurus | unknown | cattle | NMR structure of the bovine prion protein Direct |
| | | | (cattle) | | | Submission |
| 1DX1_A | 6.4 | 2000 | Bos taurus | unknown | cattle | NMR structure of the bovine prion protein Direct |
| | | | (cattle) | | | Submission |
| 2K1D_A | 6.8 | 2009 | Homo sapiens | unknown | human | Residue 129 Polymorphism And Conformational |
| | | | (human) | | | Dynamics Of Familial Prion Diseases Associated |
| | | | | | | With The Human Prion Protein Variant D178n |
| | | | | | | Direct Submission |
| 2LEJ_A | 6.8 | 2011 | Homo sapiens | unknown | human | Toward the Molecular Basis of Inherited Prion |
| | | | (human) | | | Diseases: NMR Structure of the Human Prion |
| | | | | | | Protein with V210I Mutation Direct Submission |
| NP_114468 | 7 | 2001 | Rattus | unknown | Norway | Human DnaJ homologs dj2 and dj3; and bag-1 are |
| | | | norvegicus | | rat | positive cochaperones of hsc70 Minireview: the |
| | | | (Norway rat) | | | intersection of steroid receptors with molecular |
| | | | | | | chaperones: observations and questions Induction |
| | | | | | | of molecular chaperones in carbon tetrachloride- |
| | | | | | | treated rat liver: implications in protection against |
| | | | | | | liver damage RDJ2 (DNAJA2) chaperones neural G |
| | | | | | | protein signaling pathways A human protein- |
| | | | | | | protein interaction network: a resource for |
| | | | | | | annotating the proteome Chaperoning of |
| | | | | | | glucocorticoid receptors Molecular chaperones |
| | | | | | | throughout the life cycle of the androgen receptor |
| | | | | | | Expression cloning of a novel farnesylated protein; |
| | | | | | | RDJ2; encoding a DnaJ protein homologue Rdj2; a |
| | | | | | | J protein family member; interacts with cellular |
| | | | | | | prion PrP(C) |
| 2KUN_A | 8.8 | 2010 | Homo sapiens | unknown | human | NMR structure of the human prion protein with |
| | | | (human) | | | the pathological Q212P mutation reveals unique |
| | | | | | | structural features Direct Submission |
| 1FKC_A | 9.2 | 2000 | Homo sapiens | unknown | human | Solution structure of the E200K variant of human |
| | | | (human) | | | prion protein. Implications for the mechanism of |
| | | | | | | pathogenesis in familial prion diseases Direct |
| | | | | | | Submission |
| 1FO7_A | 9.2 | 2000 | Homo sapiens | unknown | human | Solution structure of the E200K variant of human |
| | | | (human) | | | prion protein. Implications for the mechanism of |
| | | | | | | pathogenesis in familial prion diseases Direct |
| | | | | | | Submission |
| AEG75818 | 12 | 2011 | Mesocricetus | unknown | golden | Direct Submission Differential expression of |
| | | | auratus | | hamster | interferon responsive genes in rodent models of |
| | | | (golden | | | transmissible spongiform encephalopathy disease |
| | | | hamster) | | | |

### Example 11

### Vaccine Against Prion Disease

A vaccine was designed for prevention and blocking of prion disease. The vaccine may be manufactured in seven days or less. The vaccine may be shipped freeze-dried and may be administered via any method known to one of skill in the art. The vaccine is designed to inhibit initiation of the disease and to stop progression of the disease.

The vaccine comprises an approximate equal-parts-by-weight mixture of SEQ ID NO(s): 1-8. The peptides are generated using solid-phase synthesis and an equal-parts-by-weight mixture is generated in sterile water. The vaccine is administered to an animal susceptible to a prion disease. Peptides reflecting SEQ ID NO(s): 1-8 generate an immune and/or blocking response against the prion disease.

1. An immunogenic composition comprising an isolated or synthesized Replikin peptide sequence identified in a prion disease or an isolated or synthesized homologue of a Replikin peptide sequence identified in a prion disease or an isolated or synthesized antigenic fragment of said Replikin peptide sequence or homologue of said Replikin peptide sequence, wherein the Replikin peptide sequence identified in the prion disease is part of a body affected or changed by the disease, wherein said Replikin peptide sequence consists of the shortest amino acid sequence of 7 to 50 amino acid residues comprising (1) a first lysine residue located six to ten residues from a second lysine residue, (2) at least one histidine residue; and (3) at least 6% lysine residues and wherein said homologue is at least 80% homologous with said Replikin peptide sequence.

2. The immunogenic composition of claim 1, wherein the part of a body affected or changed by a prion disease is a protein, protein fragment, polypeptide, or peptide.

3. The immunogenic composition of claim 1, wherein the prion disease is Creutzfeldt-Jakob Disease, Variant Creutzfeldt-Jakob Disease, Gerstmann-Straussler-Scheinker Syndrome, Fatal Familial Insomnia, Kuru, bovine spongiform encephalopathy, chronic wasting disease, scrapie, transmissible mink encephalopathy, feline spongiform encephalopathy, or ungulate spongiform encephalopathy.

4. The immunogenic composition of claim 1, wherein said homologue of a Replikin peptide sequence is at least 90%, homologous with a Replikin peptide sequence identified in a prion disease.

5. The immunogenic composition of claim 1, wherein said homologue of a Replikin peptide sequence has the same lysine residues and histidine residue present in the Replikin peptide sequence identified in a prion disease.

6. The immunogenic composition of claim 1 comprising a peptide that consists essentially of a Replikin peptide sequence identified in a prion disease.

7. The immunogenic composition of claim 1 comprising a peptide that consists of a Replikin peptide sequence identified in a prion disease.

8. A vaccine comprising at least one immunogenic composition of claim 1.

9. The vaccine of claim 8 comprising more than one Replikin peptide sequence, homologue of a Replikin peptide sequence, peptide sequence sharing the structure of lysine residues and histidine residue that define a Replikin sequence, antigenic fragment of a Replikin peptide sequence, or antigenic fragment of a homologue of a Replikin peptide sequence.

10. The vaccine of claim 8 comprising at least one peptide sequence of SEQ ID NO(s): 1-8, at least one homologue of SEQ ID NO(s): 1-8, at least one peptide sharing the same lysine residues and histidine residue creating the Replikin structure of SEQ ID NO(s): 1-8, at least one peptide consisting essentially of any one of SEQ ID NO(s): 1-8, at least one peptide consisting of any one of SEQ ID NO(s): 1-8, or at least one antigenic fragment of at least one of SEQ ID NO(s): 1-8.

11. The vaccine of claim 8, further comprising at least one pharmaceutically-acceptable carrier, excipient, adjuvant, other additional component, or combination thereof.

12. A method of making a vaccine against a prion disease comprising identifying at least one Replikin peptide sequence or fragment of a Replikin peptide sequence in a protein, protein fragment, polypeptide, or peptide expressed in a part of a body affected or changed by the disease and making a vaccine comprising said protein, protein fragment, polypeptide, or peptide comprising said Replikin peptide sequence, a homologue of said Replikin peptide sequence, or a fragment of said Replikin peptide sequence, wherein said Replikin peptide sequence consists of the shortest amino acid sequence of 7 to 50 amino acid residues comprising (1) a first lysine residue located six to ten residues from a second lysine residue, (2) at least one histidine residue; and (3) at least 6% lysine residues and wherein said homologue is at least 80% homologous with said Replikin peptide sequence.

13. The method of making a vaccine of claim 12, wherein said vaccine comprises a plurality of Replikin peptide sequences, fragments of Replikin peptide sequences, or homologues of Replikin peptide sequences.

14. The method of making a vaccine of claim 12, wherein said vaccine comprises at least one peptide sequence of SEQ ID NO(s): 1-8, at least one fragment of SEQ ID NO(s): 1-8, or at least one homologue of SEQ ID NO(s): 1-8.

15. An isolated or synthesized peptide consisting of a Replikin peptide sequence identified in a part of a body affected or changed by a prion disease; a homologue of a Replikin peptide sequence identified in a part of a body affected or changed by a prion disease, wherein said homologue is at least 80% homologous with said Replikin peptide sequence; a peptide sharing the same lysine residues or histidine residue that define a Replikin peptide sequence identified in a part of a body affected or changed by a prion disease; or an antigenic fragment of a Replikin peptide sequence identified in a part of a body affected or changed by a prion disease.

16-17. (canceled)

18. The isolated or synthesized peptide of claim 15, wherein said Replikin sequence is at least one of SEQ ID NO(s): 1-8.

19. The isolated or synthesized peptide of claim 18 consisting essentially of at least one of SEQ ID NO(s): 1-8.

20. The isolated or synthesized peptide of claim 19 consisting of at least one of SEQ ID NO(s): 1-8.

21-25. (canceled)
